(19) United States
(12) Patent Application Publication
(10) Pub. No.: US 2002/0107173 A1
(43) Pub. Date: Aug. 8, 2002

(54) Method of treating amyloid beta precursor disorders

(76) Inventors: Lawrence Friedhoff, Hackensack, NJ (US); Joseph Buxbaum, New York, NY (US); Edward I. Cullen, Montvale, NJ (US)

Correspondence Address: DAVIDSON, DAVIDSON & KAPPEL, LLC, 14th Floor, 185 Seventh Avenue, New York, NY, 10018, US

(21) Appl. No.: 10/067,593

(22) Filed: Feb. 5, 2002

Related U.S. Application Data
(60) Provisional application No. 60/265,886, filed on Feb. 5, 2001.
(60) Provisional application No. 60/163,608, filed on Nov. 4, 1999.
(60) Provisional application No. 60/219,435, filed on Jul. 20, 2000.
(60) Provisional application No. 60/223,987, filed on Aug. 9, 2000.

(51) Int. Cl. A61K 31/401; A61K 31/366; A61K 31/22

(52) U.S. Cl. 514/1; 514/423; 514/460; 514/547

(57) ABSTRACT

Methods for the treatment and prevention of APP processing disorders such as Alzheimer's disease and Down's Syndrome which are based on the administration of an effective amount of a HMG-CoA reductase inhibitor to a mammal are disclosed. Additionally, methods for the treatment and prevention of APP processing disorders such as Alzheimer's disease and Down's Syndrome which are based on the reduction of cellular cholesterol in a mammal are disclosed. These methods reduce the amount of Aβ peptides or decrease the formation of Aβ peptides or increase the clearance of Aβ peptides in a mammal suffering from Alzheimer's disease and Down's Syndrome.

### RELATED APPLICATION DATA

This application claims priority to U.S. Provisional Patent Application No. 60/265,886 filed Feb. 5, 2001, the disclosure of which is hereby incorporated by reference. Also incorporated by reference are U.S. Provisional Patent Application No. 60/163,608, filed Nov. 4, 1999, U.S. Provisional Patent Application No. 60/219,435 filed Jul. 22, 2000 and U.S. Provisional Patent Application No. 60/223,987, filed Aug. 9, 2000.

### TECHNICAL FIELD

The present invention relates to a method of treating amyloid β precursor protein (APP) disorders such as Alzheimer's disease and Down's Syndrome.

### BACKGROUND OF THE INVENTION

The cause of Alzheimer's disease is not known. The disease is characterized by the accumulation of β-amyloid peptides (Aβ peptides), as abnormal protein precipitates, in the brain. It is generally believed that these proteins kill brain cells which causes a loss of mental function.

As illustrated in FIG. 1, immature amyloid β precursor protein (APPᵢ) under-goes glycosylation to become mature amyloid β precursor protein (APPₘ). Then APPₘ is either (1) cleaved by the protease α-secretase to produce a secreted form of APP (APPₛ) which is not amyloidogenic, or (2) cleaved by β-secretase and γ-secretase to produce the abnormal protein, Aβ (Aβ peptide), which can then precipitate.

Many advances have been made in the treatment of Alzheimer's disease. The cholinesterase inhibitors such as tacrine, donepezil and rivastigmine improve symptoms slightly. However, the slight improvement in attention and alertness is most likely due to increased brain acetylcholine levels. Unfortunately, however, the cholinesterase inhibitors do not prevent cognitive decline, which is inevitably fatal even with optimal cholinesterase inhibitor treatment.

Several strategies for treating Alzheimer's disease have been proposed and include decreasing or preventing the release of AP peptide by either increasing α-secretase or decreasing the β- or γ-secretase activity or production. Other strategies include decreasing Aβ peptide aggregation, increasing Aβ peptide clearance, reducing Aβ peptide production or decreasing the cellular effects of Aβ peptide aggregation and deposition. See Sabbagh, M. N. et al., (1997) Alzheimer's Disease Rev. 3:1-19. See also U.S. Pat. No. 6,080,778. In light of the foregoing, there is a need for a more effective treatment of mammals suffering from APP processing disorders such as Alzheimer's disease and Down's Syndrome.

### SUMMARY OF THE INVENTION

Generally, the invention relates to a method for treating a mammal having an APP processing disorder comprising administering to the mammal a composition comprising a therapeutically effective amount of at least one HMG-CoA reductase inhibitor. APP processing disorders include Alzheimer's disease and Down's Syndrome.

In a preferred embodiment, the invention relates to a method of treating a mammal having Alzheimer's disease and/or Down's Syndrome by administering to the mammal a therapeutically effective amount of at least one HMG-CoA reductase inhibitor. In this embodiment, the method may also comprises determining whether the mammal exhibits at least one objective symptom of Alzheimer's disease or Down's Syndrome.

In another embodiment of the present invention, the composition comprising at least one HMG-CoA reductase inhibitor may further comprise a pharmaceutically acceptable excipient. The composition is preferably in the form of a controlled release formulation.

In a preferred embodiment of the present invention, the HMG-CoA reductase inhibitor is selected from the group consisting of mevastatin, pravastatin, simvastatin, atorvastatin, lovastatin, rivastatin and fluvastatin, and pharmaceutically effective salts, isomers and the active metabolite forms thereof, or a combination thereof. In a more preferred embodiment, the HMG-CoA reductase inhibitor is lovastatin or lovastatin acid.

In another preferred embodiment, about 0.2 mg to about 10 mg of the HMG-CoA reductase inhibitor per Kg of the mammal's body weight per day is administered. The daily amount administered to the mammal may be administered in more than one fraction.

In another preferred embodiment, an oral dose of about 5 mg to about 400 mg of lovastatin per day is administered to a human having an APP processing disorder. In a more preferred embodiment the oral dose is about 10 mg to about 350 mg per day. More preferably, the oral dose is about 10 mg to about 300 mg per day. Even more preferably, the oral dose is about 10 mg to about 250 mg per day In alternate embodiments, the dose of lovastatin can be up to 240 mg, from about 10 mg to about 120 mg or about 10 mg to about 60 mg.

In another preferred embodiment, any suitable dose of an HMG-CoA reductase inhibitor is administered to a mammal having an APP processing disorder. More preferably, the suitable dose is one that is therapeutically effective and results in the average blood plasma concentration of the HMG-CoA reductase inhibitor or its active metabolite at steady-state being below about 50 micromolar. More preferably, the blood plasma concentration of the HMG-CoA reductase inhibitor or its active metabolite at steady-state is below about 30 micromolar. Even more preferably, the blood plasma concentration of the HMG-CoA reductase inhibitor or its active metabolite at steady-state is below about 20 micromolar. In an even more preferred embodiment, the blood plasma concentration of the HMG-CoA reductase inhibitor or its active metabolite at steady-state is below about 10 micromolar. Even more preferably, the blood plasma concentration of the HMG-CoA reductase inhibitor or its active metabolite at steady-state is below about 5 micromolar. Even more preferably, the blood plasma concentration of the HMG-CoA reductase inhibitor or its active metabolite at steady-state is below about 1 micromolar. Most preferably, the blood plasma concentration of the HMG-CoA reductase inhibitor or its active metabolite at steady-state is about 0.5 micromolar.

In another preferred embodiment, any suitable dose of an HMG-CoA reductase inhibitor is administered to a mammal having an APP processing disorder. More preferably, the suitable dose is one that is therapeutically effective and results in the average blood plasma concentration of the HMG-CoA reductase inhibitor or its active metabolite at steady-state being below about 50 nanomolar. More preferably, the blood plasma concentration of the HMG-CoA reductase inhibitor or its active metabolite at steady-state is below about 30 nanomolar. Even more preferably, the blood plasma concentration of the HMG-CoA reductase inhibitor or its active metabolite at steady-state is below about 20 nanomolar. In an even more preferred embodiment, the blood plasma concentration of the HMG-CoA reductase inhibitor or its active metabolite at steady-state is below about 10 nanomolar. Even more preferably, the blood plasma concentration of the HMG-CoA reductase inhibitor or its active metabolite at steady-state is below about 5 nanomolar. Even more preferably, the blood plasma concentration of the HMG-CoA reductase inhibitor or its active metabolite at steady-state is below about 1 nanomolar. Most preferably, the blood plasma concentration of the HMG-CoA reductase inhibitor or its active metabolite at steady-state is about 0.5 nanomolar.

In another embodiment, the invention relates to a method for treating a mammal having an APP processing disorder which comprises lowering the amount of Aβ peptides in the brain, cerebral spinal fluid, or plasma of the mammal by administering to the mammal a composition comprising a therapeutically effective amount of at least one HMG-CoA reductase inhibitor. Lowering the amount of Aβ peptides in the brain may comprise affecting APPₘ processing. In a preferred embodiment, the amount of Aβ peptides is lowered in the brain of the mammal.

In another embodiment, the invention relates to a method for treating a mammal having an APP processing disorder which comprises lowering the amount of Aβ peptides in the brain, cerebral spinal fluid, or plasma of the mammal by administering to the mammal a composition comprising a therapeutically effective amount of at least one NSAID or secretose modifier. Lowering the amount of Aβ peptides in the brain may comprise affecting APPₘ processing. In a preferred embodiment, the amount of Aβ peptides is lowered in the brain of the mammal.

In another embodiment, the invention relates to a method for treating a mammal having an APP processing disorder which comprises increasing the clearance of Aβ peptides in the brain, cerebral spinal fluid, or plasma of the mammal by administering to the mammal a composition comprising a therapeutically effective amount of at least one HMG-CoA reductase inhibitor. In a preferred embodiment, the clearance of Aβ peptides in the brain of the mammal is increased.

In another embodiment, the invention relates to a method for treating a mammal having an APP processing disorder comprising preventing or reducing Aβ peptide aggregation or plaque formation in the brain of the mammal by administering to the mammal a composition comprising a therapeutically effective amount of at least one HMG-CoA reductase inhibitor.

In another embodiment, the invention relates to a method for the treatment of a mammal exhibiting the objective symptoms of Alzheimer's disease by decreasing the formation of Aβ peptides, increasing the clearance of Aβ peptides, regulating the processing of APP, or reducing plaque maturation in the mammal by administering to the mammal a composition comprising a therapeutically effective amount of at least one HMG-CoA reductase inhibitor.

In another embodiment, the invention relates to a method for treating a mammal having an APP processing disorder comprising lowering the amount cellular cholesterol levels in the mammal. In a preferred embodiment, the amount of cellular cholesterol levels are decreased by the administration of at least one HMG-CoA reductase inhibitor.

In certain embodiments, the detected Aβ level is decreased by about 5% or more in the body fluid.

Generally an immediate release or a controlled release dosage form may be utilized in the practice of the invention. The immediate release dosage formulation may comprise an effective amount of a HMG-CoA reductase inhibitor and a suitable pharmaceutical diluent. The controlled release dosage formulation may comprise a compressed tablet core which contains an alkyl ester of a hydroxy substituted naphthalene derivative, a pharmaceutically acceptable, water swellable polymer and an osmotic agent; and an outer coating layer which covers the osmotic core and comprises a pH sensitive coating agent and a water insoluble polymer.

An optional sealing coat may be applied to the compressed tablet core and an optional coating layer comprising an enteric coating agent may be applied under the outer coating layer as an inner coating or as an overcoat over the outer coating layer. The tablet core may be compressed using a smooth faced tablet die. The preferred alkyl ester of a hydroxy substituted naphthalene compound is lovastatin. Plasma levels of about 0.5 micromoles of the HMG-CoA reductase inhibitor are preferably maintained by the use of a controlled release formulation of the HMG-CoA reductase inhibitor.

In certain embodiments, the present invention provides for a method of managing the HMG-CoA reductase inhibitor treatment of a patient with Alzheimer's disease. Preferably, the present invention provides a method for monitoring the effect of a therapeutic treatment on a subject who has undergone therapeutic treatment with an HMG-CoA reductase inhibitor. This method comprises measuring at suitable time intervals the amount of β-amyloid concentration in a body fluid. Any change or absence of change in the amount of the β-amyloid can be identified and correlated with the effect of the therapeutic treatment on the subject. In certain preferred embodiments the present invention involves detecting a change or no change in the β-amyloid levels, in the HMG-CoA reductase inhibitor therapy and adjusting the HMG-CoA reductase therapy accordingly.

In certain embodiments, the measured amount of the β-amyloid is compared to a baseline level. Preferably, this baseline level of β-amyloid concentration is the level present in the subject prior to HMG-CoA reductase inhibitor therapy. In certain embodiments, the baseline level is the level measured in a patient on existing HMG-CoA reductase inhibitor therapy.

In certain embodiments, the invention is directed to a method for managing a patient with Alzheimer's disease or at risk of developing Alzheimer's disease comprising providing to said patient a therapeutic agent which lowers Aβ levels, and detecting a level of Aβ in a body fluid of said patient to determine the efficacy of said therapeutic agent. In further embodiments, the invention further comprises repeatedly detecting the level of Aβ in a body fluid and/or repeatedly providing said therapeutic agent according to a dosing interval (e.g., once or twice daily).

In certain embodiments, the invention comprises comparing a detected level of Aβ in said body fluid with at least one previously detected level of Aβ in order to determine the efficacy of the therapeutic agent. The detected level can also be compared to an accepted value known in the art which is accepted as normal or indicative of the disease state. In further embodiments, the invention comprises adjusting the repeated dosing of said therapeutic agent based on said comparison.

Any procedures known in the art for the measurement of β-amyloid levels can be used in the practice of the instant invention. Such procedures include but are not limited to competitive and non-competitive assay systems using techniques such as radioimmunoassays, ELISA (enzyme linked immunosorbent assay), “sandwich” immunoassays, precipitin reactions, gel diffusion precipitin reactions, immunodiffusion assays, agglutination assays, complement-fixation assays, immunoradiometric assays, fluorescent immunoassays, western blots, protein A immunoassays, and immunoelectro-phoresis assays, combinations thereof and the like. Generally speaking, the method for quantitative measurement of involves capture of the β-amyloid with a first capture-antibody, washing away all unbound components, and detecting the remaining complex with a second detection-antibody. Preferably, the immunoassay designs are based on numerous “capture and detection-antibody” combinations, and may involve combinations of antibodies, provided that each antibody reacts with separate epitopes. Preferably the method comprises using Aβ peptide antibodies to capture and detect the presence of Aβ peptide in the body fluid.

In certain preferred embodiments, ELISA (enzyme linked immunosorbent assay) can be used. One description of such an embodiment is for example as follows: A monoclonal antibody (capture antibody, mAb 1) directed against the soluble antigen is adsorbed onto a solid substratum. The soluble antigen present in the sample binds to the antibody, and unreacted sample components are removed by washing. An enzyme-conjugated monoclonal antibody (detection antibody, mAb 2) directed against a second epitope of the antigen binds to the antigen captured by mAb 1 and completes the sandwich. After removal of unbound mAb 2 by washing, a substrate solution is added to the wells. In certain embodiments, a colored product is formed in proportion to the amount of antigen present in the sample. The reaction is terminated by addition of stop solution and absorbance may be measured spectrophotometrically or, in some embodiments, the product may be detected Fluorometrically.

In preferred embodiments, the antibodies for use in the present invention are specific only for Aβ peptide.

In certain embodiments, the assay method of the present invention can be provided in the form of a kit, e.g., a packaged combination of instructions for carrying out the assay, capture antibody, and solid support for immobilization as described hereinafter. In addition, a detection means may also be included, such as an antibody to the Aβ peptide, which may be labeled or unlabeled, as well as other additives, such as for example, stabilizers, washing, and incubation buffers, and the like.

Kits of the present invention, also will typically include a means for containing the reagents in close confinement for commercial sale such as, e.g., injection or blow-molded plastic containers. Other containers suitable for conducting certain steps of the disclosed methods also may be provided.

### BRIEF DESCRIPTION OF THE DRAWINGS

FIG. 1 is a schematic which illustrates APP processing.

FIGS. 2a and 2b illustrate the effects of lovastatin acid on Aβ peptides in Human Neuroglioma (H4) cells. FIG. 2a is a photograph of two gel wells wherein the negative well and the positive well correspond to the bar graphs of FIG. 2b representing 0 and 0.5 μM of lovastatin acid, respectively. Data represent the mean±the standard error of the mean (SEM) of one experiment performed in quadruplicate.

FIGS. 3a and 3b illustrate the effects of lovastatin acid on Aβ peptides in Madin-Darby Canine Kidney (MDCK) cells. FIG. 3a is a photograph of two gel wells wherein the negative well and the positive well correspond to the bar graphs of FIG. 3b representing 0 and 0.5 μM of lovastatin acid, respectively. Data represent the mean±SEM of three experiments performed in quadruplicate.

FIGS. 4a and 4b illustrate the effects of lovastatin acid on Aβ peptides in Chinese Hamster Ovary (CHO) cells. FIG. 4a is a photograph of two gel wells wherein the negative well and the positive well correspond to the bar graphs of FIG. 4b representing 0 and 0.5 μM of lovastatin acid, respectively. Data represent the mean±SEM of four experiments performed in quadruplicate.

FIGS. 5 illustrates the effects of lovastatin acid on APPₛ processing. Data represent the mean±SEM of an experiment performed in quadruplicate.

FIG. 6 illustrates the effects of lovastatin acid on mature APP processing. Data represent the mean±SEM of an experiment performed in quadruplicate.

FIG. 7 is a graph showing the steady-state plasma concentrations of lovastatin acid in patients after multiple oral 40 mg doses of Lovastatin XL, a preferred extended release tablet form of lovastatin.

FIG. 8 is a graph showing the change in the mean Aβ peptide concentration in the blood of groups of patients after treatment with various doses of Lovastatin XL.

FIG. 9 is a bar chart showing the change in the mean Aβ peptide concentration in the blood of groups of patients after treatment with various doses of Lovastatin XL.

### DETAILED DESCRIPTION OF THE INVENTION

Recently, the present inventors have discovered that HMG-CoA reductase inhibitors lower the amount of Aβ peptide levels, prevent or reduce Aβ peptide formation, may increase Aβ clearance, and therefore prevent or reduce Aβ peptide aggregation. More particularly, the present inventors have discovered that the administration of HMG-CoA reductase inhibitors lower the amount of Aβ peptide levels, prevent or reduce Aβ peptide formation, may increase Aβ clearance, and therefore prevent or reduce Aβ peptide aggregation, without the need of other cholesterol lowering treatments. Therefore, methods of treating APP processing disorders such as Alzheimer's disease and Down's Syndrome in a mammal comprising the administration of a HMG-CoA reductase inhibitor to the mammal is disclosed herein below.

As used herein, “APPᵢ” means the immature form of amyloid β protein precursor, “APPₘ” means the mature form of amyloid β protein precursor, “APPₛ” means the amyloid β protein precursor which is cleaved by α-secretase and which is the secreted form, “APP” means either APPᵢ, APPₘ, or both.

As used herein, “post-translational” events include the cleavage of APPₘ by β- and γ-secreatases.

As used herein, the term “body fluid” refers to a biological sample of liquid containing the Aβ peptide. Such fluids include aqueous fluids such as serum, plasma, lymph fluid, synovial fluid, follicular fluid, seminal fluid, amniotic fluid, milk, whole blood, urine, cerebro-spinal fluid, saliva, sputum, tears, perspiration, mucus, tissue culture medium, tissue extracts, and cellular extracts.

As used herein, “other cholesterol lowering treatments” means any treatment other than treatment with a HMG-CoA reductase inhibitor. Other cholesterol lowering treatments include, but are not limited to, treatment with mevalonate, methyl-β-cyclodextrin, and/or cyclodextrin.

As used herein, “active metabolite” is intended to mean a pharmacologically active product produced through metabolism in the body of a specified compound or salt thereof. Active metabolites of a compound may be identified using routine techniques known in the art. See, e.g., Bertolini, G. et al., J. Med. Chem., 40, 2011-2016 (1997); Shan, D. et al., J. Pharm. Sci., 86 (7), 765-767; Bagshawe K., Drug Dev. Res., 34, 220-230 (1995); Bodor, N., Advances in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

As used herein, the term “pharmaceutically acceptable salts” refers to salt forms that are pharmacologically acceptable and substantially non-toxic to the subject being administered the composition of the present invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and famaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from organic bases such as amines, benzylamines, piperidines, and pyrrolidines.

For purposes of the present invention, the term “managing a patient” means monitoring at least one detected level of Aβ in a patient on existing therapy of a therapeutic agent which lowers Aβ or after initiation of such therapy, in order to obtain an indicator of the progression or inhibition of the disease state. The term may also include providing the patient with drug therapy, as well as adjustments of further doses of therapy.

For purposes of the present invention, the term “providing to a patient a therapeutic agent” means taking action which results in the initiation or continuation of therapy with a therapeutic agent (e.g., administering an agent to a patient, prescribing an agent for a patient, instructing a patient to continue existing therapy, etc.).

Any HMG-CoA reductase inhibitor may be used in the method of the present invention. Alternatively, any NSAID or secretose modifier may also be used in the method of the present invention. The term “HMG-CoA reductase inhibitor” refers to any one or more compounds that inhibit the bioconversion of hydroxymethylglutamyl-coenzyme A to mevalonic acid which is catalyzed by the enzyme HMG-CoA reductase. Such inhibition may be determined by standard methods known to those of ordinary skill in the art. Examples of suitable HMG-CoA reductase inhibitors are described and referenced herein, however, other HMG-CoA reductase inhibitors will be known to those of ordinary skill in the art. Therefore, the present invention should not be limited to the specific HMG-CoA reductase inhibitors exemplified herein.

Examples of such HMG-CoA reductase inhibitors which are useful in the method of the present invention for the treatment of Alzheimer's disease include mevastatin which is described in U.S. Pat. No. 3,671,523; lovastatin which is described in U.S. Pat. No. 4,231,938; pravastatin which is described in U.S. Pat. No. 4,346,217; simvastatin which is described in U.S. Pat. No. 4,444,784; atorvastatin which is described in U.S. Pat. No. 4,647,576; rivastatin which is described in European Pat. No. 491226A; and fluvastatin which is described in U.S. Pat. No. 4,739,073. All of these patents are incorporated herein by reference. Further, any suitable isomers of the exemplified HMG-CoA reductase inhibitors may be used, including stereoisomers, enantiomers, or mixtures thereof and, thus, their use in pharmaceutical formulations for the treatment of APP disorders are within the scope of the invention.

Lovastatin is a metabolite which is produced by the natural fermentation of a fungus of the Aspergillus genus. The other compounds of this class are derived from natural and synthetic sources using well known procedures and have similar mechanisms of activity.

Any suitable NSAID known in the art can be used in the present invention, including but not limited to the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen and tolmetin. The salicylates may include acetylsalicylic acid, sodium acetylsalicylic acid, calcium acetylsalicylic acid, salicylic acid, and sodium salicylate.

Any suitable method for administering the HMG-CoA reductase inhibitor may be used. For example, the HMG-CoA reductase inhibitors may be administered orally to a mammal having Alzheimer's disease or Down's Syndrome in an effective amount to relieve the symptoms of Alzheimer's disease or Down's Syndrome.

Preferably, the effective amount of the HMG-CoA reductase inhibitor results in the average blood plasma concentrations of the HMG-CoA reductase inhibitor or its active metabolite at steady-state being below about 50 micromolar. More preferably, the blood plasma concentration of the HMG-CoA reductase inhibitor or its active metabolite at steady-state is below about 30 micromolar. Even more preferably, the blood plasma concentration of the HMG-CoA reductase inhibitor or its active metabolite at steady-state is below about 20 micromolar. In an even more preferred embodiment, the blood plasma concentration of the HMG-CoA reductase inhibitor or its active metabolite at steady-state is below about 10 micromolar. Even more preferably, the blood plasma concentration of the HMG-CoA reductase inhibitor or its active metabolite at steady-state is below about 5 micromolar. Even more preferably, the blood plasma concentration of the HMG-CoA reductase inhibitor or its active metabolite at steady-state is below about 1 micromolar. Most preferably, the blood plasma concentration of the HMG-CoA reductase inhibitor or its active metabolite at steady-state is about 0.5 micromolar.

In another embodiment, the effective amount preferably of the HMG-CoA reductase inhibitor results in the average blood plasma concentrations of the HMG-CoA reductase inhibitor or its active metabolite at steady-state being below about 50 nanomolar. More preferably, the blood plasma concentration of the HMG-CoA reductase inhibitor or its active metabolite at steady-state is below about 30 nanomolar. Even more preferably, the blood plasma concentration of the HMG-CoA reductase inhibitor or its active metabolite at steady-state is below about 20 nanomolar. In an even more preferred embodiment, the blood plasma concentration of the HMG-CoA reductase inhibitor or its active metabolite at steady-state is below about 10 nanomolar. Even more preferably, the blood plasma concentration of the HMG-CoA reductase inhibitor or its active metabolite at steady-state is below about 5 nanomolar. Even more preferably, the blood plasma concentration of the HMG-CoA reductase inhibitor or its active metabolite at steady-state is below about 1 nanomolar. Most preferably, the blood plasma concentration of the HMG-CoA reductase inhibitor or its active metabolite at steady-state is about 0.5 nanomolar.

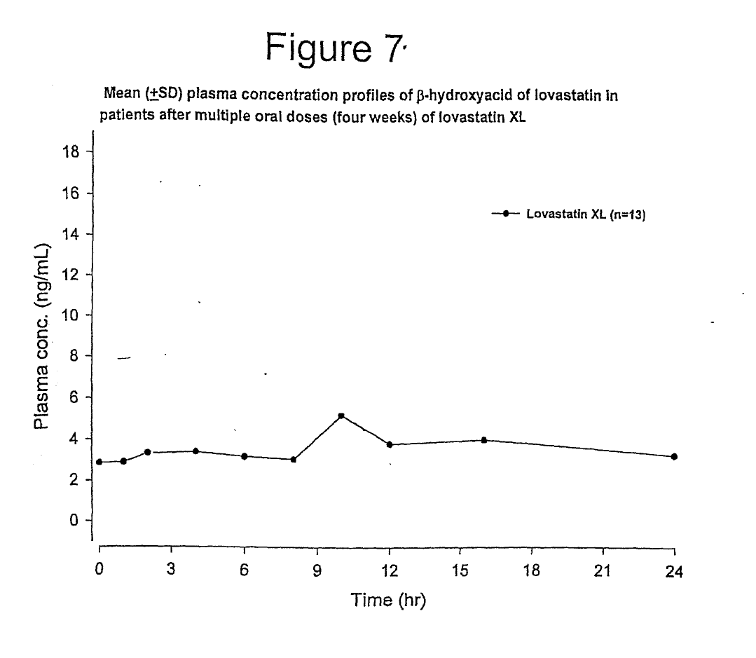

FIG. 7 shows the steady-state plasma concentrations (nanograms/ml) of lovastatin acid in patients after multiple oral 40 mg doses of Lovastatin XL, a preferred extended release tablet form of lovastatin. Accordingly, based on a conversion factor and the known linear pharmacokinetics of lovastatin it can be expected that oral doses of about 233 mg Lovastatin XL (“Lovastatin XL” refers to a lovastatin controlled release formulation as exemplified herein below) given daily to a patient would result in average blood plasma level of the patient being about 0.05 micromolar.

However, the present inventors have surprisingly discovered that human patients given oral doses of only 10 mg/day, 20 mg/day, 40 mg/day or 60 mg/day of Lovastatin XL resulted in a statistically significant decrease in Aβ peptide levels in the blood plasma of those patients. Accordingly, the inventors have unexpectedly found that the HMG-CoA reductase inhibitor may be administered to a human orally at daily doses of about 10 mg to about 60 mg.

Preferably, the HMG-CoA reductase inhibitor is administered to the mammal orally at a daily dose of about 0.2 mg to 10.0 mg per kg of body weight. The HMG-CoA reductase inhibitors may be administered in any suitable form. For example, the HMG-CoA reductase inhibitor may be administered in the form of tablets, capsules or oral concentrates suitable for mixing the particular compound with food.

The criteria for the diagnosis of Alzheimer's disease is well known and is set forth in the guidelines of the National Institute of Neurological and Communicative Disorders and Alzheimer's Disease and Related Disorders Association (McKhann et al., Neurology 1984: 34: 939-944); and in the American Psychiatric Association, Diagnostic and Statistical Manual of Mental Disorders (Diagnostic and Statistical Manual IV), all of which are incorporated herein by reference. Generally the objective criteria for the diagnosis of Alzheimer's disease include: gradual memory impairment and gradual onset of at least one of the following aphasia, apraxia, agnosia or disturbance of executive functioning.

Treatment may be continued until there is a reduction in the symptoms of Alzheimer's disease and the dosage may be adjusted in response to the mammal's individual response. Generally a positive response will not be expected until therapy has been continued for a minimum period of 90 to 365 days.

More preferably, a controlled release formulation (also herein after referred to as a “controlled release composition”) of the HMG-CoA reductase inhibitor is utilized in order to provide an enhanced effect that cannot be achieved by conventional immediate release dosing. The use of a controlled release form may be specially useful for providing a constant level of the HMG-CoA reductase inhibitor in order to avoid dosage peaks and valleys in those mammals who have meals at irregular times or those who frequently eat snacks between meals.

Controlled release formulations have been described in U.S. Pat. No. 4,615,698 which have been based on an osmotic dosage form which is designed to collapse and cause the faced surfaces to come into a closed contacting arrangement as the drug is delivered through a passageway in the semi-permeable wall of the dosage form. In addition, U.S. Pat. No. 4,503,030 discloses an osmotic dosage form which has a passageway and a semi-permeable membrane consisting of a particular cellulose polymer and a pH sensitive material which could be an enteric coating material. This patent describes the use of 1:1 mixtures of a pH sensitive material and cellulose polymer which are applied at a level of about 7% by weight based on the total weight of the osmotic core tablet and coating material. The aforementioned patents are incorporated herein by reference.

### Preferred HMG-CoA Reductase Inhibitor Formulations

A preferred controlled release formulation is disclosed in U.S. Pat. No. 5,916,595, which is incorporated herein by reference. This type of a controlled release dosage form is preferably prepared by combining the HMG-CoA reductase inhibitor with a pharmaceutically acceptable, water swellable polymer and an osmotic agent into a compressed tablet core having an optional first coating for sealing and protection and a second coating comprising a pH sensitive agent water insoluble polymer. More preferably, the HMG-CoA reductase inhibitor is selected from the group consisting of mevastatin, pravastatin, simvastatin, atorvastatin, and lovastatin and the active metabolite forms thereof. Even more preferably, the HMG-CoA reductase inhibitor comprises lovastatin or its active metabolite, lovastatin acid. Mevastatin, pravastatin, simvastatin, atorvastatin, and lovastatin are well known compounds that are described in the prior art including the particular patents which have been cited herein. It is also within the scope of the invention to use mixtures of different alkyl esters of hydroxy substituted naphthalenes.

Specifically, the pharmaceutically acceptable, water swellable polymer and the osmotic agent are combined with the HMG-CoA reductase inhibitor which may be micronized, comicronized or unmicronized or amorphous or crystalline and compressed to form the tablet core. The osmotic agent is any suitable non-toxic pharmaceutically acceptable water soluble compound which will dissolve sufficiently in water and increase the osmotic pressure inside the simple sugars and salts such as sodium chloride, potassium chloride, magnesium sulfate, magnesium chloride, sodium sulfate, lithium sulfate, urea, inositol, sucrose, lactose, glucose, sorbitol, fructose, mannitol, dextrose, magnesium succinate, potassium acid phosphate and the like. The preferred osmotic agent for the tablet core is a simple sugar such as anhydrous lactose in the range of about 0-50% by weight, based on the weight of the compressed, uncoated tablet.

The pharmaceutically acceptable, water swellable polymer may be any pharmaceutically acceptable polymer which swells and expands in the presence of water to slowly release the HMG-CoA reductase inhibitor. These polymers include polyethylene oxide, methylcellulose, hydroxypropylcellulose, hydroxypropylmethylcellulose and the like.

In a preferred embodiment, the water swellable polymer will be polyethylene oxide (obtained from Union Carbide Corporation under the trade name Polyox WSR Coagulant or Polyox WSR N 80). These materials form a viscous gel in water or other solvent system at a sufficient concentration to control the release of the HMG-CoA reductase inhibitor. This will generally require a concentration of the pharmaceutically acceptable water swellable polymer of about 0-50% by weight of the compressed, uncoated tablet.

Any suitable binder may be employed. Preferably, the binder is used in a sufficient amount so that when it is combined with a suitable solvent, mixed with the water soluble osmotic agent and agitated, granules will be formed which may be compressed into a tablet core. Prior to compressing the granules, the conventional solid pharmaceutical diluents such as microcrystalline cellulose, lactose, dextrose and the like may be added to the granule based on the weight of the compressed, uncoated tablet. In the present case, the above mentioned osmotic agent, lactose, may function as a binder in the tablet compression step.

In the preparation of the tablets, any suitable solvent may be used to prepare the aforementioned granules. In addition, various other suitable diluents, excipients, lubricants, dyes, pigments, dispersants, emulsifiers, and the like may be used to optimize the HMG-CoA reductase inhibitor formulation.

Additionally, any suitable surfactant may be used. The surfactant may be any ionic or non-ionic water soluble surfactant which is preferably employed in the range of about 0-50% by weight and more preferably employed in the range of about 1-5% by weight. The preferred surfactant for the present formulation is sodium lauryl sulfate but other surfactants such as polysorbate 20, 60, or 80; polyoxl 40 stearate and the like may be used.

Furthermore, a tabletizing formulation may also include any suitable lubricant. Ideally, the lubricant will be in the range of from about 0.5 to about 2.5% by weight of the compressed, uncoated tablet.

After the above described tablet core is formed, it is preferably coated with: 1) an optional protective first coating on the tablet core and/or an optional pH sensitive coating; and 2) an outer coating comprising a pH sensitive agent and a water insoluble polymer.

Specifically, a protective first coating may be used at a level in the range of about 0-10% by weight which may be applied from a coating system such as OPADRY CLEAR™ sold by Colorcon Corporation. In an especially preferred embodiment, the OPADRY CLEAR™ will be about 2.83% by weight and will be combined with an osmotic agent in the range of about 0-10% by weight. While the osmotic agent may be any suitable salt, low molecular weight molecule or water soluble polymer, the preferred osmotic agent is sodium chloride. Preferably, the osmotic agent is added to the coating system when the coating system is being dispersed into purified water. The coating system which contains the osmotic agent may then be sprayed onto the tablets to form a protective coating layer.

An optional inner or over coat over the outer coat may also be applied which comprises a pH sensitive polymer which functions as an enteric polymer in that it does not begin to dissolve until pH conditions in excess of the stomach region are encountered. Generally, the pH sensitive materials do not dissolve and begin to release the active drug until the pH is about 3.0, and preferably above about 5.5. Materials such as Eudragit L (copolymer of poly(methacrylic acid, methylmethacrylate), 1:1 ratio; MW (No. Av. 135,000—USP Type A) or Eudragit S (copolymer of poly(methacrylic acid, methylmethacrylate, 1:2 ratio MW (No. Av. 135,000—USP Type B) may be used. Hydroxypropyl methyl cellulose phthalate and the like may be used in the range of about 0-30% by weight and preferably about 2 to about 4% by weight of the combined weight of the compressed, uncoated tablet and the inner coating of the pH sensitive polymer.

Preferably, the outer coating comprises a pH sensitive polymer which functions as an enteric polymer in that it does not begin to dissolve until pH conditions in excess of the pH of the stomach region are encountered and a water insoluble polymer which provide controlled release properties to the coating formulation. The pH sensitive polymer is preferably the same type of material that is described above as the optional inner coating layer. The water insoluble polymer may be a cellulosic polymer such as ethylcellulose, cellulose acrylate, cellulose mono-, di- or triacetate. The pH sensitive polymer and the insoluble cellulosic polymer are used at a weight ratio of about 0.1:1 to about 0.75:1, preferably about 0.25:1 to about 0.5:1 of pH sensitive polymer to water insoluble cellulosic polymer. A combined coating weight of about 0.5-5% by weight and preferably about 1-4% by weight and especially preferred is about 1-3% by weight of the gained weight based on the weight of the coated tablet core. Cellulose acetate is the preferred water insoluble polymer and the outer coating is preferably applied as a suspension in acetone.

Furthermore, any suitable plasticizer or combination of plasticizers may be added to the inner, outer or over coating to provide elasticity and shape to the coating. The plasticizer or combination of plasticizers may be any water soluble or water insoluble formulation in the range of about 0-10% by weight and preferably about 0.5-5% by weight of the outer coating composition. Acetyltributyl citrate is the preferred plasticizer but material such as acetyl triethyl citrate, dibutyl phthalate, triacetin, diethyl phthalate, polyethylene glycol, propylene glycol and the like may be utilized.

Any suitable antioxidant such as butylated hydroxyanisole (BHA) or butylated hydroxytoluene (BHT) may be added to the tablet core as a stabilizer at a level of about 0.001-0.01 % by weight of the tablet core.

Any suitable channeling agent may be mixed with the aforementioned components of the outer coating. A channeling agent may be employed to increase the porosity of the film coating in order to increase the amount of the fluids that penetrate the tablet core and increase the rate of hydration. This allows the release of the HMG-CoA reductase inhibitor after the outer film coat ruptures. Generally, channeling agents may be any salts, surfactants, or short-chain water soluble polymers in a water channel forming effective amount, i.e., about 1-5% by weight, based on the total weight of the core and all coating components. The channeling agents include any pharmaceutically acceptable water soluble salt, surfactant, or short-chain water soluble polymer such as sodium chloride, potassium chloride, sucrose, polysorbate-8O, hydroxypropyl cellulose, hydroxyethyl cellulose and the like.

Also, the inner or over coating may be supplied with an anti-sticking agent such as talc to overcome any tablet to tablet stickiness during the coating process. The amount of anti-sticking agent supplied is preferably in an amount which prevents sticking, more preferably in the range of about 0-6% by weight based on the weight of the tablets and the coating materials on a dry weight basis.

The tablets may be made by any suitable method, for example, in a smooth faced tablet die. Thereafter the tablet is preferably provided with the outer coating, which because of surface tension, will result in a thinner coating layer over the comers of the tablet which will provide an area in the outer coating which will form a channel to allow intestinal fluid to reach the core of the tablet.

As used herein, the term “antibodies” includes all types of immunoglobulin molecules, monoclonal antibodies, polyclonal antibodies, affinity-purified polyclonal antibodies, Fab and (Fab)₂, single-chain (SC) antibodies, or other molecules which specifically bind an epitope on Aβ. Such antibodies are produced in accordance with known techniques in the art. Generally, the antibodies used to detect Aβ according to this invention will be labeled with a detectable label, such as a radiolabel, a fluorescent label, second antibody specific for a separate epitope on Aβ antibody where the second antibody is conjugated to an enzyme that is used to catalyze the production of a detectable signal.

Any antibody which specifically binds to an epitope on Aβ is potentially useful in the assays of this invention. Examples of such antibodies include for example and without limitation, two antibodies (pAb 1-17 and pAb 17-28) to residues 1-17 and 17-28 of Aβ made by Quality Controlled Biochemicals Inc. (Hopkinton, Mass.), and Ab 6E10 to Aβ 1-17 and mAb 4G8 to Aβ 17-24, commercially available from Senetek, and antibodies described in U.S. Pat. No. 5,955,317, to Suzuki et al, the disclosure of which is hereby incorporated by reference. Many suitable techniques for using such antibodies to detect Aβ epitopes will be apparent to the skilled artisan, including fluorescence activated cell sorting (FACS), sandwich assays, competitive immunoassays, ELISA assays, Western blots, dot blots, ouchterlony plates, immunoelectrophoresis, fluorimetry, microcopy, fluorescence microscopy, ultra-filtration (using radiolabeled antibodies) and others.

In a preferred embodiments, the body fluid is analyzed by ELISA using antibodies specific for epitopes on Aβ. An ELISA apparatus typically comprises a 96 well microtiter plate, the inside surfaces of which are coated with one of the Aβ specific antibodies. This coating, binding or attachment of the antibody to the solid phase is not a chemical reaction but rather is believed to result from a physical or noncovalent interaction between the polystyrene matrix of the microtiter plate and the antibody. A sample suspected of containing the target molecule Aβ is placed in contact with the coated microtiter plate so that binding will occur between the ligand Aβ in the sample and the antibody. Any unbound components in the sample fluid are then removed from the plate wells by several washing steps. A second antibody which specifically recognizes the target molecule and is linked to a signal-generating enzyme is then added. Detection of the enzyme which is indicative of the presence of the target molecule in the sample is typically performed by addition of reagents which produce a detectable signal such as fluorescence or a color change.

In accordance with the present invention, preferably the body fluid is contacted and incubated with an immobilized capture antibody. The solid phase used for immobilization may be any inert support or carrier that is preferably water insoluble and useful in immunometric assays, including supports in the form of, e.g., surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96-well microtiter plates, as well as particulate materials such as filter paper, agarose, cross-linked dextran, and other polysaccharides. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture reagent immobilization. The preferred solid phase used is a multi-well microtiter plate that can be used to analyze several samples at one time. The most preferred is a microtest 96-well ELISA plate such as that sold as Nunc Maxisorb or Immulon. The solid phase is coated with the capture reagent as defined above, which may be linked by a non-covalent or covalent interaction or physical linkage as desired. If 96-well plates are utilized, they are preferably coated with the capture antibody and incubated for at least about 10 hours, more preferably at least overnight.

The coated plates are then typically treated with a blocking agent that binds non specifically to and saturates the binding sites to prevent unwanted binding of the detection antibody to the excess non-specific sites on the surfaces of the wells of the plate. Examples of appropriate blocking agents for this purpose include, e.g., gelatin, bovine serum albumin, egg albumin, casein, and non-fat milk. After coating and blocking, the body fluid to be analyzed, appropriately diluted, is added to the immobilized phase.

The conditions for incubation of sample and immobilized capture reagent are selected to optimize sensitivity of the assay. Usually constant temperatures are maintained during the incubation period. Various buffers may be employed to achieve and maintain the desired pH during this step, including borate, phosphate, carbonate, Tris-HCl or Tris-phosphate, citrate, acetate, barbital, and the like. The particular buffer employed is not critical to the invention, but in individual assays one buffer may be preferred over another.

The body fluid is separated (preferably by washing) from the immobilized capture antibody to remove uncaptured body fluid. The solution used for washing is generally a buffer (“washing buffer”) with a pH that will depend on the capture reagent utilized. The washing may be done one or more times.

In the last step of the assay method, the Aβ that is now bound to the capture antibody is measured. This measurement may be accomplished by many techniques, such as extraction to remove the bound Aβ from the capture reagent followed by bioassay, radioreceptor assay, or radioimmunoassay.

More preferably, however, the amount of free ligand is analyzed in the same plate, without the need for extraction or other cumbersome steps, using a standard ELISA method as detection means. In this procedure, preferably a molar excess of an antibody with respect to the maximum concentration of Aβ expected is added to the plate after it is washed.

The detection antibody added to the immobilized capture antibody will be either directly labeled, or detected indirectly by addition, after washing off of excess first antibody, of a molar excess of a second, labeled antibody directed against the first detection antibody.

The label used for either the first or second detection antibody is any detectable functionality that does not interfere with the binding of free ligand to the antibody. Examples of suitable labels are those numerous labels known for use in immunoassay, including moieties that may be detected directly, such as fluorochrome, chemiluminscent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptide. Preferred labels herein are enzymes such as horseradish peroxidase and alkaline phosphatase.

Following the addition of last labeled antibody, the amount of bound antibody is determined by removing excess unbound labeled antibody through washing and then measuring the amount of the attached label using a detection method appropriate to the label, and correlating the measured amount with the amount of Aβ in the body fluid.

As a matter of convenience, the assay method of this invention can be provided in the form of a kit, i.e., a packaged combination of instructions for carrying out the assay, capture reagent as defined above, antibodies, standards for the Aβ, and solid support for immobilization as defined above. In addition, a detection means as defined above may be included, such as a specific antibody to the Aβ, which is labeled or unlabeled, as well as other , additives such as stabilizers, washing and incubation buffers, and the like.

A preferred control release tablet useful in the practice of the present invention will have the following general formula as set forth in Table 1:

TABLE 1

| Tablet Core: | |
|---|---|
| Alkyl ester of a substitute naphthalene | 3-20 wt % |
| Water Swellable Polymer | 10-40 wt % |
| Antioxidant | 0.001-0.01 wt % |
| Osmotic Agents | 20-80 wt % |
| Surfactant | 0-5 wt % |
| Lubricant | 0-5 wt % |
| Coatings: | |
| Seal Coating | 0-10 wt % |
| Osmotic Agents | 0-10 wt % |
| Inner Coating: | |
| Enteric Polymer | 0-30 wt % |
| Anti-sticking Agent | 0-6 wt % |
| Plasticizer | 0-6 wt % |
| Channeling Agents | 0-6 wt % |
| Outer Coating: | |
| Blend of Enteric Polymer and Water- | 0.5-5 wt % |
| insoluble Polymer | |
| Plasticizer(s) | 0-1 wt % |
| Channeling Agents | 0.2-5 wt % |
| Overcoat: | |
| Enteric Polymer | 0-30 wt % |
| Anti-sticking Agent | 0-6 wt % |
| Plasticizer | 0-6 wt % |
| Channeling Agents | 0-6 wt % |
| TOTAL | 100 wt % |

A particularly preferred tablet which is useful in the practice of the invention has the ingredients as set forth in Table 2 and may be prepared as set forth below:

TABLE 2

| | | |
|---|---|---|
| Lovastatin | 12.14 wt % | 20.00 mg |
| Polyox WSR Coagulant, NF (polyethylene | 4.55 wt % | 7.50 mg |
| oxide Mw No. AV 5,000,000) | | |
| Polyox WSR N 80, NF (polyethylene oxide | 17.76 wt % | 29.25 mg |
| Mw No. AV 200,000) | | |
| Lactose (anhydrous) | 51.30 wt % | 84.50 mg |
| Sodium lauryl sulfate | 3.04 wt % | 5.00 mg |
| Cab-O-Sil (Silicon dioxide Fumed US/NF) | 0.46 wt % | 0.75 mg |
| Butylated hydroxy anisole | 0.03 wt % | 0.05 mg |
| Myvaplex 600P (glyceryl monostearate) | 1.82 wt % | 3.00 mg |
| Seal Coating: | | |
| Opadry Clear (mixture containing | 3.42 wt % | 5.63 mg |
| hydroxypropyl methyl cellulose and | | |
| polyethylene glycol) | | |
| Sodium Chloride | 1.14 wt % | 1.88 mg |
| Outer Coating: | | |
| Cellulose acetate | 1.43 wt % | 2.36 mg |
| Eudragit S 100 (poly(methylacrylic acid) | 0.49 wt % | 0.80 mg |
| methylacrylate) 1:2 ratio MW (No. Av. | | |
| 135,000 - USP Type B) | | |
| Triacetin (Glycerol Triacetate) | 0.11 wt % | 0.19 mg |
| Polyethylene glycol 400 | 0.11 wt % | 0.19 mg |
| Sugar, confectioners 6X micronized | 0.72 wt % | 1.18 mg |
| Overcoat: | | |
| Hydroxypropylmethylcell. Phthal. 55 | 0.77 wt % | 1.27 mg |
| Talc | 0.30 wt % | 0.49 mg |
| Acetyl tributyl citrate | 0.12 wt % | 0.20 mg |
| Sugar, confectioners 6X micronized | 0.30 wt % | 0.49 mg |
| TOTAL | 100.0 wt % | 146.73 mg |

The following describes the preferred process of making the above described dosage form:

Step 1. The Tablet Core

(a) Granulation

1. Pass Polyox WSR N80, sodium lauryl sulfate and anhydrous lactose through a 30 mesh stainless steel screen.

2. Charge the screened materials and lovastatin (micronized) into a vertical granulator.

3. Prepare a butylated hydroxy anisole solution by dissolving butylated hydroxy anisole in ethanol.

4. Prepare a mixture of ethanol and purified water.

5. Pre-mix the powder mixture from above (step 1 (a)2) for 5 minutes.

6. Blend the powder mixture again, add the butylated hydroxyanisole solution and then the ethanol/water mixture.

7. Dry the resulting granules at 45-50° C. until the moisture content is lower than 1.8 wt %.

8. Pass the granules through a 1575 mesh using a Comil.

(b) Tabletting

1. Mix Cab-O-Sil and Polyox WSR N80.

2. Pass the mixture of Cab-O-Sil and Polyox WSR N80 through a 24 mesh stainless steel screen with the Polyox WSR Coagulant.

3. Blend the screened materials with lovastatin granules for 15 minutes.

4. Pass Myvaplex through a 30 mesh stainless steel screen and combine with the other screen materials.

5. Blend for five minutes.

6. Compress the blend into tablets (164.72 mg, round, standard concave, 17/64″ dia.) which contain 20 mg of lovastatin.

(c) Seal Coating: Opadry Clear

1. Dissolve sodium chloride in purified water.

2. Disperse Opadry Clear into the sodium chloride solution.

3. Spray lovastatin tablets with the aqueous coating suspension using a coater.

(d) Inner Coating: None

(e) Outer Coating: Cellulose Acetate

1. Dissolve cellulose acetate and Eudragit S100 in acetone using a homogenizer.

2. Add polyethylene glycol 400, triacetein and sugar to the solution and mix until a homogenous dispersion is obtained.

3. Spray the coating suspension onto the tablets in a coater.

(f) Overcoating: Hydroxypropyl Methylcellulose Phthalene 55 (HPMCP 55)

1. Dissolve hydroxypropyl methylcellulose phthalene 55 in acetone using a homogenizer.

2. Add acetyl tributyl citrate to the acetone solution and mix it with a homogenizer until a homogenized dispersion is obtained.

3. Add talc and sugar to the solution and mix it with a homogenizer until a homogenized dispersion is obtained.

4. Replace the homogenizer with a magnetic mixer and stir the coating mixture throughout the coating process.

5. Spray the Opadry Clear coated lovastatin tablets with the coating dispersion in a coater.

Other particularly preferred control release tablets useful in the practice of the present invention are those disclosed in U.S. patent application Ser. No. 09/435,576, which is herein incorporated by reference.

For example, a particularly preferred tablet which is useful in the practice of the present invention has the ingredients as set forth in Table 3 and may be prepared as set forth below:

TABLE 3

| | | |
|---|---|---|
| Lovastatin | 11.99 wt % | 40.0 mg |
| Polyox WSR Coagulant, | 4.50 wt % | 15.0 mg |
| NF (polyethylene oxide | | |
| Mw No. AV 5,000,000) | | |
| Polyox WSR N 80, | 17.98 wt % | 60.0 mg |
| NF (polyethylene oxide Mw | | |
| No. AV 200,000) | | |
| Lactose (anhydrous) | 50.65 wt % | 169.0 mg |
| Sodium lauryl sulfate | 3.00 wt % | 10.0 mg |
| Silicon dioxide Fumed USP/NF | 0.45 wt % | 1.5 mg |
| Myvaplex 600P (glyceryl monostearate) | 1.80 wt % | 6.0 mg |
| Seal Coating: | | |
| Opadry Clear (mixture | 2.81 wt % | 9.4 mg |
| containing hydroxypropyl | | |
| methyl cellulose and polyethylene glycol | | |
| Sodium Chloride | 0.93 wt % | 3.1 mg |
| Inner Coating: | | |
| Hydroxypropylmethylcell.phthal.55 | 2.27 wt % | 7.58 mg |
| Talc | 0.78 wt % | 2.60 mg |
| Acetyl tributyl citrate | 0.22 wt % | 0.75 mg |
| Sugar, confectioners 6X micronized | 0.62 wt % | 2.08 mg |
| Outer Coating: | | |
| Cellulose acetate | 1.00 wt % | 3.32 mg |
| Eudragit S 100 (poly(methacrylic acid), | 0.34 wt % | 1.13 mg |
| methylmetharcrylate, 1:2 ratio MW (No. Av. | | |
| 135,000-USP Type B) | | |
| Triacetin (Glycol Triacetate) | 0.08 wt % | 0.27 mg |
| Polyethylene glycol 400 | 0.08 wt % | 0.27 mg |
| Sugar, confectioners 6X micronized | 0.50 wt % | 1.66 mg |
| TOTAL | 100.00 wt % | 333.66 mg |

The following describes a suitable process of making the above described dosage form:

Granulation

1. Pass Polyox WSR N80, sodium lauryl sulfate and anhydrous lactose through a 30 mesh stainless steel screen.

2. Charge the screened materials and lovastatin (micronized) into a vertical granulator.

3. Dissolve buylated hydroxy anisole in ethanol.

4. Mix ethanol, and purified water.

5. Pre-mix the powder mixture for 5 minutes.

6. Blend the powder mixture again, add the butylated hydroxyanisole solution and then the ethanol/water mixture.

7. Dry the granules at 45-50° C. until the moisture content is lower than 1.8 wt %.

8. Pass the granules through a 1575 mesh using a Comil.

Tabletting

1. Mix Cab-O-Sil and Polyox WSR N80.

2. Pass the mixture of Cab-O-Sil and Polyvox WSR N80 through a 24 mesh stainless steel screen with the Polyox WSR Coagulant.

3. Blend the screen materials with lovastatin granules for 15 minutes.

4. Pass Myvaplex through a 30 mesh stainless steel screen and combine with the other screen materials.

5. Blend for five minutes.

6. Compress the blend into tablets (300 mg, round standard concave, 11/32″) which contain 40 mg of lovastatin.

Seal Coating: Opadry Clear

1. Dissolve sodium chloride in purified water.

2. Disperse Opadry Clear into the sodium chloride solution.

3. Spray lovastatin tablets with the aqueous coating suspension using a coater.

Inner Coating: Hydroxypropyl Methylcellulose Phthalate 55

1. Dissolve hydroxypropyl methylcellulose phthalate 55 in acetone using a homogenizer.

2. Add acetyl tributyl citrate to the acetone solution and mix it with a homogenizer until a homogenized dispersion is obtained.

3. Add talc and sugar to the solution and mix it with a homogenizer until a homogenized dispersion is obtained.

4. Replace the homogenizer with a magnetic mixer and stir the coating mixture throughout the coating process.

5. Spray the Opadry Clear coated lovastatin tablets with the coating dispersion in a coater.

Outer Coating: Cellulose Acetate

1. Dissolve cellulose acetate and Eudragit S100 in acetone using a homogenizer.

2. Add polyethylene glycol 400, triactein and sugar to the solution and mix until a homogeneous dispersion is obtained.

3. Spray the coating suspension onto the tablets in a coater.

Another example of a particularly preferred tablet has the ingredients as set forth in Table 4:

TABLE 4

| | | |
|---|---|---|
| Lovastatin | 12.11 wt % | 40.0 mg |
| Polyox WSR Coagulant, | 4.54 wt % | 15.0 mg |
| NF (polyethylene oxide MW No | | |
| av. 5,000,000) | | |
| Polyox WSR N 80, | 17.71 wt % | 58.5 mg |
| NF (polyethylene oxide Mw No av | | |
| 200,000) | | |
| Lactose (anhydrous) | 51.13 wt % | 168.9 mg |
| Sodium lauryl sulfate | 3.03 wt % | 10.0 mg |
| Cab-O-Sil (Silicon dioxide Fumed USP/NF) | 0.45 wt % | 1.5 mg |
| Butylated hydroxy anisole | 0.03 wt % | 0.10 mg |
| Myvaplex 600P (glyceryl monostearate) | 1.82 wt % | 6.0 mg |
| Seal Coating: | | |
| Opadry Clear (mixture | 2.85 wt % | 9.4 mg |
| containing hydroxypropyl methyl | | |
| cellulose and polyethylene glycol) | | |
| Sodium Chloride | 0.94 wt % | 3.1 mg |
| Inner Coating: | | |
| Hydroxypropylmethylcell.phthal.55 | 2.29 wt % | 7.58 mg |
| Talc | 0.79 wt % | 2.6 mg |
| Acetyl tributyl citrate | 0.23 wt % | 0.75 mg |
| Sugar, confectioners 6X micronized | 0.08 wt % | 0.27 mg |
| Outer Coating: | | |
| Cellulose acetate | 1.00 wt % | 3.32 mg |
| Eudragit S 100 (polymethacrylicacid, | 0.34 wt % | 1.13 mg |
| methylmethaceylate, | | |
| 1:2 ratio MW (No. AV. 135,000- | | |
| USP Type B) | | |
| Triacetin | 0.08 wt % | 0.27 mg |
| Polyethylene glycol 400 | 0.08 wt % | 0.27 mg |
| Sugar, confectioners 6X micronized | 0.50 wt % | 1.66 mg |
| TOTAL | 100.00 wt % | 330.35 mg |

The preferred tablet having the ingredients as set forth in Table 4 may be prepared as described above for the preparation of the preferred tablet having the ingredients as set forth in Table 3.

Another example of a particularly preferred tablet has the ingredients as set forth in Table 5 and may be prepared as set forth below:

TABLE 5

| | | |
|---|---|---|
| Lovastatin | 12.14 wt % | 20.0 mg |
| Polyox WSR Coagulant, NF (polyethylene | 4.55 wt % | 7.5 mg |
| oxide Mw No. AV 5,000,000) | | |
| Polyox WSR N 80, NF (polyethylene oxide | 17.76 wt % | 29.25 mg |
| Mw No. AV 200,000) | | |
| Lactose (anhydrous) | 51.30 wt % | 84.5 mg |
| Sodium lauryl sulfate | 3.04 wt % | 5.0 mg |
| Cab-O-Sil (Silicon dioxide Fumed USP/NF) | 0.46 wt % | 0.75 mg |
| Butylated hydroxy anisole | 0.03 wt % | 0.05 mg |
| Myvaplex 600P (glyceryl monostearate) | 1.82 wt % | 3.0 mg |
| Seal Coating: | | |
| Opadry Clear (mixture containing | 3.42 wt % | 5.63 mg |
| hydroxypropyl methyl cellulose and | | |
| polyethylene glycol) | | |
| Sodium chloride | 1.14 wt % | 1.88 mg |
| Outer Coating: | | |
| Cellulose acetate | 1.43 wt % | 2.36 mg |
| Eudragit S 100 (polymethylacrylic acid, | 0.49 wt % | 0.80 mg |
| methylacrylate, 1:2 ratio MW (No. Av. | | |
| 135,000 - USP Type B) | | |
| Triacetin | 0.11 wt % | 0.19 mg |
| Polyethylene glycol 400 | 0.11 wt % | 0.19 mg |
| Sugar, confectioners 6X micronized | 0.72 wt % | 1.18 mg |
| Overcoat: | | |
| Hydroxypropylmethylcell.Phthal.55 | 0.77 wt % | 1.27 mg |
| Talc | 0.30 wt % | 0.49 mg |
| Triacetin | 0.12 wt % | 0.20 mg |
| Sugar, confectioners 6X micronized | 0.30 wt % | 0.49 mg |
| TOTAL | 100.0 wt % | 146.73 mg |

The following describes the process of making the above described dosage form:

Granulation

1. Pass Polyox WSR N80, sodium lauryl sulfate and anhydrous lactose through a 30 mesh stainless steel screen.

2. Charge the screened materials and lovastatin (micronized) into a vertical granulator.

3. Dissolve butylated hydroxy anisole in ethanol.

4. Mix ethanol and purified water.

5. Pre-mix the powder mixture for 5 minutes.

6. Blend the powder mixture again, add the butylated hydroxyanisole solution and then the ethanol/water mixture.

7. Dry the granules at 45-50° C. until the moisture content is lower than 1.8 wt %.

8. Pass the granules through a 1575 mesh using a Comil.

Tabletting

1. Mix Cab-O-Sil and Polyox WSR N80.

2. Pass the mixture of Cab-O-Sil and Polyox WSR N80 through a 24 mesh stainless steel screen with the Polyox WSR Coagulant.

3. Blend the screen materials with lovastatin granules for 15 minutes.

4. Pass Myvaplex through a 30 mesh stainless steel screen and combine with the other screen materials.

5. Blend for five minutes.

6. Compress the blend into tablets (164.72 mg, round, standard concave, 17/6411 dia.) which contain 20 mg of lovastatin.

Seal Coating: Opadry Clear

1. Dissolve sodium chloride in purified water.

2. Disperse Opadry Clear into the sodium chloride solution.

3. Spray lovastatin tablets with the aqueous coating suspension using a coater.

Inner Coating: None

Outer Coating: Cellulose Acetate

1. Dissolve cellulose acetate and Eudragit S100 in acetone using a homogenizer.

2. Add polyethylene glycol 400, triactein and sugar to the solution and mix until a homogeneous dispersion is obtained.

3. Spray the coating suspension onto the tablets in a coater.

Overcoating: Hydroxypropyl Methylcellulose Phthalate 55

1. Dissolve hydroxypropyl methylcellulose phthalate 55 in acetone using a homogenizer.

2. Add acetyl tributyl citrate to the acetone solution and mix it with a homogenizer until a homogenized dispersion is obtained.

3. Add talc and sugar to the solution and mix it with a homogenizer until a homogenized dispersion is obtained.

4. Replace the homogenizer with a magnetic mixer and stir the coating mixture throughout the coating process.

5. Spray the Opadry Clear coated lovastatin tablets with the coating dispersion in a coater.

Another example of a particularly preferred tablet has the ingredients as set forth in Table 6 and may be prepared by the same general procedure as described above for the preparation of the tablet having the ingredients as set forth in Table 5, except that no inner coating is applied and an outer enteric coating is applied as an overcoat over the outer layer.

TABLE 6

| | | |
|---|---|---|
| Lovastatin | 12.20 wt % | 20.0 mg |
| Polyox WSR Coagulant, | 4.57 wt % | 7.5 mg |
| NF (Polyethylene oxide Mw | | |
| No av 5,000,000) | | |
| Polyox WSR N 80, | 17.84 wt % | 29.25 mg |
| NF (polyethylene oxide Mw No | | |
| av 200,000) | | |
| Lactose (anhydrous) | 51.53 wt % | 84.5 mg |
| Sodium lauryl sulfate | 3.05 wt % | 5.0 mg |
| Silicon dioxide fumed USP/NF | 0.46 wt % | 0.75 mg |
| Butylated hydroxy anisole | 0.03 wt % | 0.05 mg |
| Myvaplex 600P (glycerol monostearate) | 1.83 wt % | 3.0 mg |
| Seal Coating: | | |
| Opadry Clear (mixture | 3.43 wt % | 5.63 mg |
| containing hydroxypropyl | | |
| methyl cellulose and polyethylene glycol) | | |
| Sodium chloride | 1.15 wt % | 1.88 mg |
| Inner Coating: None | | |
| Outer Coating: | | |
| Cellulose acetate | 1.96 wt % | 3.21 mg |
| Eudragit S 100 | 0.66 wt % | 1.09 mg |
| Acetyl tributyl citrate | 0.32 wt % | 0.52 mg |
| Sugar: confectioners 6X micronized | 0.98 wt % | 1.61 mg |
| TOTAL | 100.00 wt % | 163.99 mg |

Other examples of particularly preferred 40 mg tablets have the ingredients as set forth in Table 7 and may be prepared by the same method described above for preparing the tablet having the ingredients as set forth in Table 3.

TABLE 7
Summary of Lovastatin Formulations

| | Weight Percent | | |
|---|---|---|---|
| Ingredient | Tablet A | Tablet B | Tablet C |
| Lovastatin (strength, mg) | 40 wt % | 40 mg | 40 mg |
| Tablet Core | | | |
| 1. Lovastatin | 12.11 wt % | 12.28 wt % | 12.28 wt % |
| 2. Lactose (Anhydrous) | 51.13 wt % | 51.8 wt % | 51.8 wt % |
| 3. Polyox ® WSR Coagulant | 4.54 wt % | 4.6 wt % | 4.6 wt % |
| 4. Polyox ® WSR N80 | 17.71 wt % | 17.94 wt % | 17.94 wt % |
| 5. Sodium Lauryl Sulfate | 3.03 wt % | 3.06 wt % | 3.06 wt % |
| 6. Glyceryl Monostearate | 1.82 wt % | 1.84 wt % | 1.84 wt % |
| 7. Silicon Dioxide | 0.45 wt % | 0.46 wt % | 0.46 wt % |
| 8. Butylated Hydroxyanisole | 0.03 wt % | 0.02 wt % | 0.02 wt % |
| Seal Coat | | | |
| 1. Opadry Clear | 2.85 wt % | 2.88 wt % | 2.88 wt % |
| 2. Sodium Chloride Powder | 0.94 wt % | 0.96 wt % | 0.96 wt % |
| Inner Coat | | | |
| 1. HPMCP55 | 2.29 wt % | 1.61 wt % | 1.61 wt % |
| 2. Talc, USP | 0.79 wt % | 0.55 wt % | 0.55 wt % |
| 3. Acetyltributyl Citrate | 0.23 wt % | 0.16 wt % | 0.16 wt % |
| 4. Sugar, Micronized | 0.64 wt % | 0.44 wt % | 0.44 wt % |
| Outer Coat | | | |
| 1. Cellulose Acetate | 1 wt % | 0.7 wt % | 0.7 wt % |
| 2. Eudragit S100 | 0.34 wt % | 0.24 wt % | 0.24 wt % |
| 3. Triacetin | 0.08 wt % | 0.06 wt % | 0.06 wt % |
| 4. Polyethylene Glycol 400 | 0.08 wt % | 0.6 wt % | 0.6 wt % |
| 5. Acetyltributyl Citrate | — | — | — |
| 6. Sugar, Micronized | 0.5 wt % | 0.35 wt % | 0.35 wt % |
| Over Coat | | | |
| 1. HPMCP 55 | — | — | — |
| 2. Talc, USP | — | — | — |
| 3. Triacetin | — | — | — |
| 4. Sugar, Micronized | — | — | — |
| 5. Opadry Yellow | — | — | — |
| 6. Opadry Pink | — | — | — |
| TOTAL TABLET | 100 wt % | 100 wt % | 100 wt % |
| WEIGHT, % | | | |

Examples of other preferred tablets having the ingredients as set forth in Table 8 may be prepared by the same method described above for preparing the tablet having the ingredients as set forth in Table 3.

TABLE 8
Summary of Lovastatin Formulations

| | Weight Percent | |
|---|---|---|
| Ingredient | Tablet D | Tablet E |
| Lovastatin (strength, mg) | 20 mg | 10 mg |
| Tablet Core | | |
| 1. Lovastatin | 11.69 wt % | 5.84 wt % |
| 2. Lactose (Anhydrous) | 49.32 wt % | 55.18 wt % |
| 3. Polyox ® WSR Coagulant | 4.38 wt % | 4.38 wt % |
| 4. Polyox ® WSR N80 | 18.08 wt % | 17.09 wt % |
| 5. Sodium Lauryl Sulfate | 2.92 wt % | 2.92 wt % |
| 6. Blyceryl Monostearate | 1.75 wt % | 1.75 wt % |
| 7. Silicon Dioxide | 0.44 wt % | 0.44 wt % |
| 8. Butylated Hydroxyanisole | 0.02 wt % | 0.01 wt % |
| Seal Coat | | |
| 1. Opadry Clear | 2.74 wt % | 2.74 wt % |
| 2. Sodium Chloride Powder | 0.91 wt % | 0.91 wt % |
| Inner Coat | | |
| 1. HPMCP 55 | 2.21 wt % | 2.21 wt % |
| 2. Talc, USP | 0.76 wt % | 0.76 wt % |
| 3. Acetyltributyl Citrate | 0.22 wt % | 0.22 wt % |
| 4. Sugar, Micronized | 0.61 wt % | 0.61 wt % |
| Outer Coat | | |
| 1. Cellulose Acetate | 0.97 wt % | 0.97 wt % |
| 2. Eudragit S100 | 0.33 wt % | 0.33 wt % |
| 3. Triacetin | 0.08 wt % | 0.08 wt % |
| 4. Polyethylene Glycol 400 | 0.08 wt % | 0.08 wt % |
| 5. Acetyltributyl Citrate | — | — |
| 6. Sugar, Micronized | 0.49 wt % | 0.49 wt % |
| Over Coat | | |
| 1. HPMCP 55 | — | — |
| 2. Talc, USP | — | — |
| 3. Triacetin | — | — |
| 4. Sugar, Micronized | — | — |
| 5. Opadry Yellow | 3 wt % | 3 wt % |
| 6. Opadry Pink | — | — |
| TOTAL TABLET WEIGHT, % | 100 wt % | 100 wt % |

As illustrated in the following examples, treatment with an HMG-CoA reductase inhibitor with concomitant cholesterol depletion may lead to a decrease in the release and formation of Aβ peptides in the cells. Additionally, the applicants have discovered that the decreased release of Aβ peptides is not due to the accumulation of the Aβ peptide in the cells, but rather due to the decreased formation of Aβ peptides. Further, the formation of APP, is also reduced by treatment with an HMG-CoA reductase inhibitor, but to a much lesser degree. Further, decreased maturation (glycosylation and sulfation) of APPᵢ has been excluded as a cause for the effects of an HMG-CoA reductase inhibitor on APPₘ processing and Aβ peptide formation. Thus, applicants have discovered that by the use of an HMG-CoA reductase inhibitor regulates APPₘ processing and Aβ formation.

In the following examples, EasyTag™ EXPRESS™ Methionine Protein Labeling Mix, [³⁵S] (spec. activity>1,000 Ci/mMol) was obtained from NEN Life Sciences, Boston, Mass.; Fetal Calf Lipid Depleted Serum (FCLPDS) was obtained from Intracel, Rockville, Md.; Dulbecco's modified Eagles Medium (DMEM) was obtained form BioWittaker, Walkersville, Md.; Dulbecco's phosphate buffered saline (PBS) and Fetal Bovine Serum (FBS) were obtained from Life Technologies, Rockville, Md.; Antibody 6EIO was obtained from Senetek, Napa, Calif.; Agarose bound antisera anti-mouse IgG was obtained from American Qualex Antibodies, San Clemente, Calif.: Protein A sepharose was obtained from Pharmacia Biotech, Piscataway, N.J.; Tissue culture plates were obtained from Falcon, Lincoln Park, N.J. with the exception of the 10 mm culture dishes with glass coverslips which were obtained from MatTek Corporation, Ashland, N. Mex.; and all other chemicals were obtained from Sigma, St. Louis, Mo.

The three cell lines utilized were: Chinese Hamster Ovary (CHO) cells expressing the 751 amino acid form of APP; Mabin-Darby Canine Kidney (MDCK) cells which overexpress the 695 amino acid form of APP; Human neuroglioma (H4) cells overexpressing the 695 form of human APP. All cells were prepared by the stable introduction of a cDNA coding for human APP. All cell lines were maintained in DMEM containing 10% FBS and antibiotics.

### EXAMPLES

The following examples are intended to illustrate but not to limit the invention.

### Example 1

### Effects of Cholesterol Depletion

In order to characterize the effects of cholesterol depletion, cell cultures of each cell line were cultured on six-well plates for 4 days in DMEM containing 10% FCLPDS, in the presence of lovastatin or lovastatin acid (LA).

In order to confirm that this treatment was as sufficient to reduce cellular cholesterol, filipin, a fluorescent dye that binds to cholesterol, was utilized to provide a visual and quantitative measure of the level of cholesterol in the membrane. Following incubation, cells were washed once with PBS and fixed with 3% paraformaldehyde in PBS for 1 hour, followed by washing 3 times in PBS for 5 minutes and quenching with 1.5 mg/ml glycine in PBS for 10 minutes. The cells were subsequently stained with 0.5 mg/ml filipin in PBS for 2 hours and washed 3 times for 5 minutes in PBS. After the final wash, the cells were visualized under a fluorescent microscope.

For measurement of APP processing and Aβ peptide formation, medium was removed and the cells were washed once with PBS and then incubated for 2 hours in DMEM containing 1 mCi/ml [³⁵S] Methionine. After this “pulse” period, the cells were either (1) lysed to measure the total labeled APPᵢ and APPₘ at time zero, or (2) the cells were incubated for 2 hours in fresh, unlabeled complete medium (“chase”) and then lysed. Then the cell supernatants and lysates were treated with the appropriate antibody to calculate the amounts of APPᵢ, APPₘ, APPₛ, and Aβ peptides.

To measure cell-associated, full-length APPᵢ and APPₘ, or to measure carboxyl-terminal fragments of APP, cell lysates were incubated with antibody 369 which recognizes the carboxyl-terminus of APP. See Buxbaum, J. D., et al. (1990) Proc Natl Acad Sci USA 87:6003-6, which is incorporated herein by reference.

To measure Aβ peptides, or to measure APPₛ, which is the secreted carboxyl-terminal truncated form, cell supernatants were incubated with antibody 6E10, which recognizes the first 15 amino acids of the Aβ peptide that correspond to the COOH-terminal amino acids of APPₛ. See Buxbaum, J. D., et al. (1994) Proc Natl Acad Sci USA, 91:4489-93, which is incorporated herein by reference.

The incubations with antibody 6E10 or antibody 369 were performed at 4° C. for 75 minutes followed by a 45 minute incubation at 4° C. with either agarose-linked anti-mouse IgG for antibody 6E1O or protein A sepharose for antibody 369. The beads were then washed three times for 10 minutes and then run on either a 10-20% Tris-Tricine Gel for APPₛ and Aβ peptides or an 8% polyacrylamide gel for cell-associated APP. The gels were dried and exposed to a Phosphor Imager® screen (STORM 860, Molecular Dynamics) and exposed for a minimum of two days. The protein bands were visualized on a STORM 860 Phosphor Imager® (Molecular Dynamics) and quantitated using ImageQuant® (Molecular Dynamics).

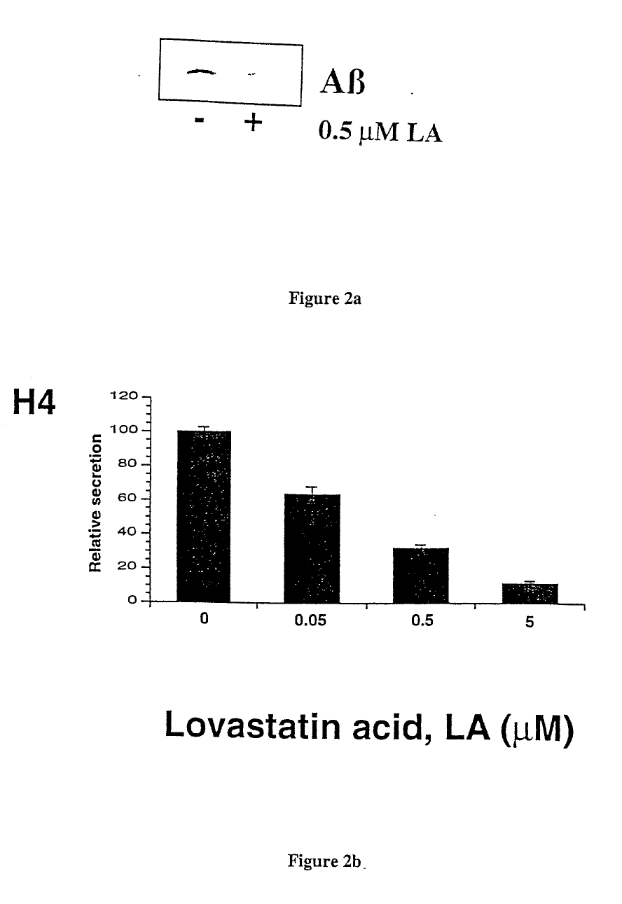

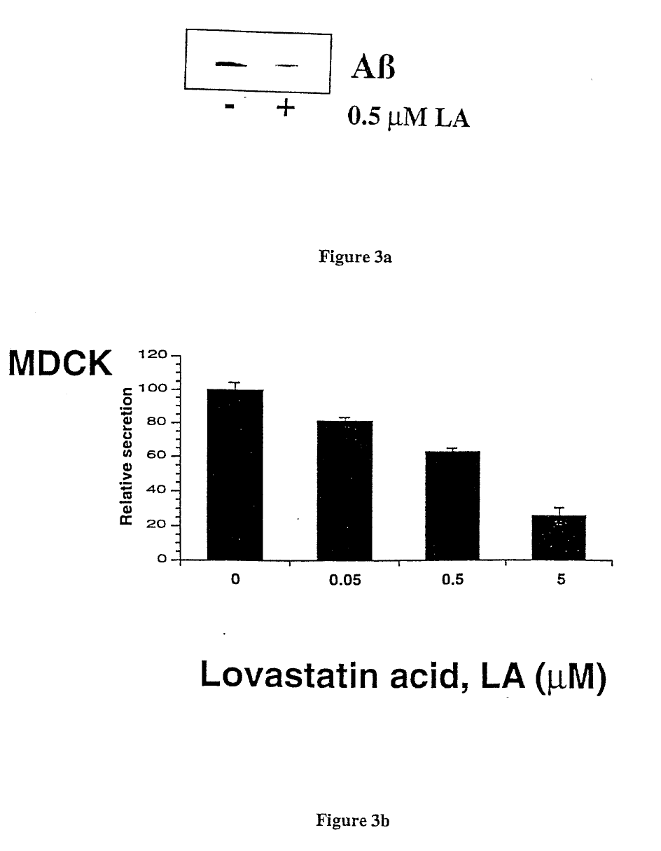

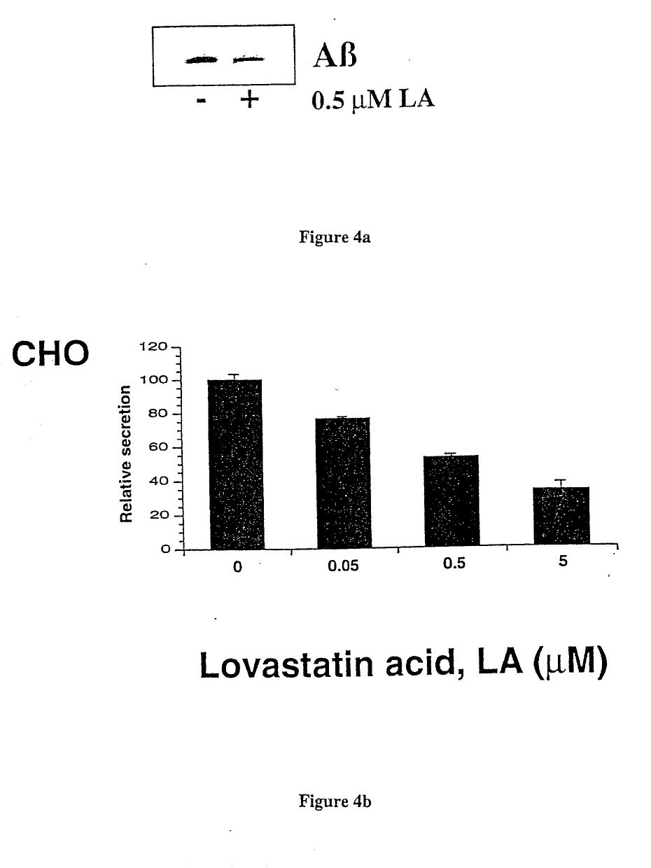

To determine whether a decrease in cholesterol is associated with a change in the amount of extracellular Aβ peptides, the H4, MDCK, and CHO cells, expressing human APPₘ were incubated for 4 days in the presence or absence of 0.5 μM LA. The cells were then incubated in serum-free media containing 1 mCi/ml [³⁵S] Methionine for 2 hours followed by incubation in complete fresh serum-free medium containing unlabeled ethionine for an additional 2 hours. The [³⁵S]-labeled Aβ peptides were immunoprecipitated from the cell culture supernatant, resolved by SDS-PAGE, and visualized by autoradiography. See FIGS. 2a, 3a, and 4a. Relative levels of extracellular Aβ peptides were determined under each condition by quantitative PhosphorImager autoradiography. See FIGS. 2b, 3b, and 4b.

By subsequently focusing only on the [³⁵S]-labeled protein, the amounts of [³⁵S]-labeled Aβ peptides were normalized to the levels of total [³⁵S]-labeled APPᵢ by dividing total [³⁵S]-labeled Aβ by total [³⁵S]-labeled APPₗ to exclude any changes in Aβ peptide levels due to decreased synthesis of Aβ peptides. At the end of the 2 hour chase the analysis was restricted to the proteolysis of APPₘ and secretion of Aβ peptides. See Buxbaum, J. D., et al. (1990) Proc Natl Acad Sci USA. 87:6003-6, which is herein incorporated by reference.

As shown in FIGS. 2a, 2b, 3a, 3b, 4a, and 4b, the amounts of extracellular Aβ peptides in the presence of 0.5 μM LA decreased by 40-60% as compared to the untreated cells. Treatment of cells with 0.5 μM lovastatin had a weaker effect on decreasing extracellular Aβ peptide levels (<20% reduction).

To determine whether the decreased levels of extracellular Aβ peptides observed were due to decreased formation of Aβ peptides, rather than decreased secretion of Aβ peptides from the cells, the levels of [³⁵S]-labeled intracellular Aβ peptides within the cell were measured in the cell lysates. No detectable levels of intracellular Aβ peptides were observed in cells incubated in the presence or absence of 0.5 μM LA. Therefore, the decrease in extracellular Aβ peptides was not due to decreased secretion of Aβ peptides, but instead confirms that it was due to the decreased formation of Aβ peptides from APPₘ.

To determine whether cholesterol depletion affects other aspects of APP processing, H4 cells were incubated for four days in the presence or absence of 0.5 μM LA, and subjected to metabolic labeling. The levels of [³⁵S]-labeled APPᵢ and APPₘ were determined by immunoprecipitation from cell lysates with an antibody against the COOH-terminal of APP, followed by quantitative autoradiography. Similarly, the levels of [³⁵S]-labeled extracellular and intracellular Aβ peptides were determined by immunoprecipitation of either cell culture supernatants (extracellular Aβ peptides) or cell lysates (intracellular Aβ peptides). The amounts of each were normalized to the levels of [³⁵S]-labeled APPᵢ found in cells at the beginning of the chase. Normalization was done by dividing the relevant value by the levels of [³⁵S]-labeled APPₗ found in cells at the beginning of the chase.

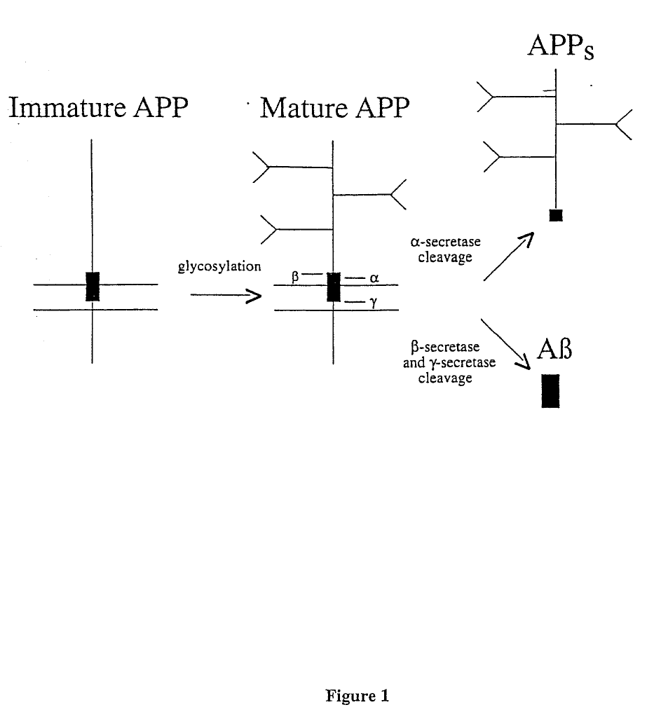

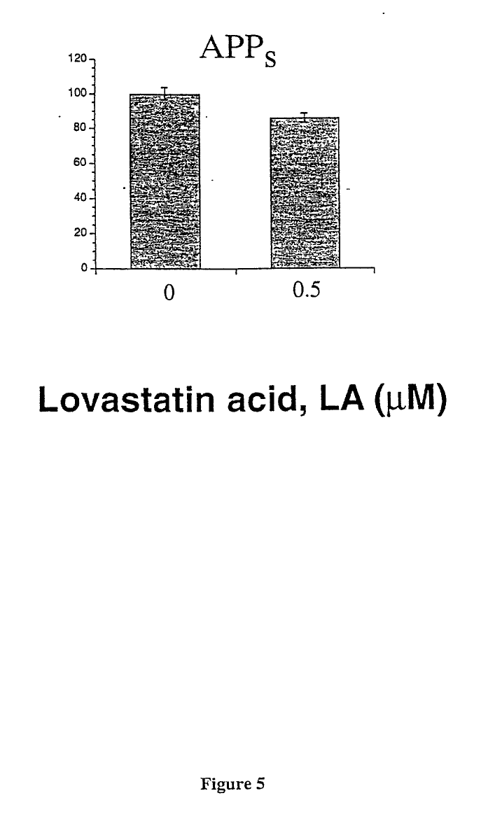

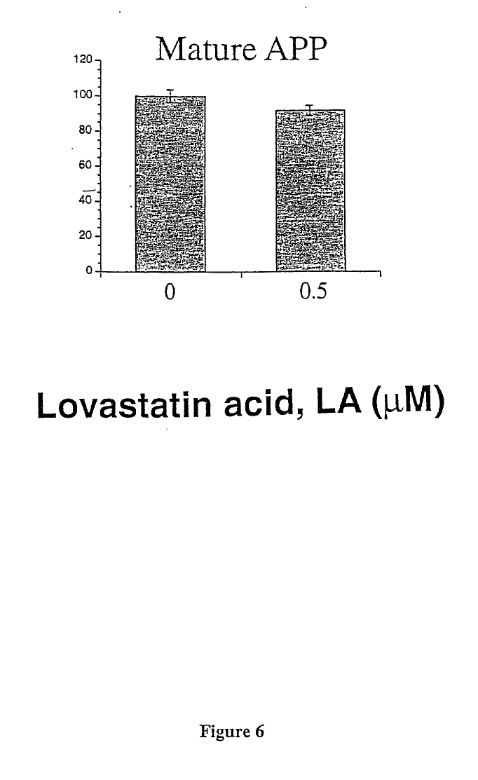

As illustrated in FIG. 5, a modest decrease in [³⁵S]-labeled APPₛ formation in cells incubated in the presence of LA, as compared to control cells was observed. FIG. 1 is a schematic illustrating APP processing. Because APPₘ is likely to be the precursor for both APPₛ and Aβ peptides, decreased formation of both APPₛ and Aβ peptides might suggest a decrease in the levels of APPₘ. To examine this, the levels of [³⁵S]-labeled APPₘ was measured in cells incubated in the absence or presence of 0.5 μM LA. As shown in FIG. 6, the effects on maturation were not sufficient to account for the decrease in Aβ peptide levels. Therefore, the effects of LA on Aβ peptide levels could not be accounted for by decreased maturation of APPₗ and instead reflect effects of LA on the post-Golgi processing or trafficking of APPₘ or both.

### Example 2

### Effective Concentration Range of Lovastatin Acid

To determine the effective range of concentrations of LA, each cell type was grown in the absence or presence of various concentrations of LA by the methods described in Example 1. As shown in the bar graphs in FIGS. 2a, 2b, 3a, 3b, 4a, and 4b, the amount of extracellular Aβ peptides decreased with increasing LA concentrations and a concentration of 0.05 μM LA or higher was sufficient to significantly (p<0.001) decrease the amount of extracellular Aβ peptides under these experimental conditions.

### Example 3

### Candidate Substance Screening

CHO cells were determined to be suitable for candidate screening because treatment with LA does not affect the maturation of APPᵢ to APPₘ in the CHO cells. Specifically, it was determined that treatment of CHO cells with 0.5 μM LA reduced the amount of extracellular APPₛ by about 30% of the amount calculated for the control and reduced the amount of extracellular Aβ peptides by about 70% of the amount calculated for the control when no LA was present. The amount of extracellular Aβ peptides and the amount of extracellular APPₛ were normalized to the amount of total APP found in the cell at the end of cell labeling as described above. This normalization provides an effective means of accounting for any differences between cultures and any differences due to altered APPₗ synthesis or maturation in cells treated with the candidate compounds. However, the level of total APPₘ was comparable between the control and the treated cells (3.2×10⁶ arbitrary units and 3.4×10⁶ arbitrary units, respectively). This suggests that the maturation of APPₗ to APPₘ was not affected in the CHO cells under experimental conditions.

Thus, CHO cells and other cells which manufacture Aβ peptides may be used as a suitable screening tool for a candidate substance which affects the synthesis, maturation or post-translational processing of APP. The cells are cultured on six-well plates for 4 days in DMEM containing 10% FCLPDS, which lipid depleted medium reduced the external source of cholesterol, in the presence of the candidate substance or absence of the candidate substance.

Specifically, the CHO cells are pulsed with [³⁵S] Methionine in the absence and presence of the candidate substance. After the pulse period, the cells are either (1) chased for two hours, or (2) lysed to determine the total intracellular APP at time zero.

Then the lysates may be labeled with the appropriate antibody to calculate the amounts of APPᵢ, APPₘ, APPₛ, and Aβ peptides.

To measure cell-associated, full-length APPᵢ and APPₘ, or to measure carboxyl-terminal fragments of APP, cell lysates are incubated with antibody 369 which recognizes the carboxyl-terminus of APP. See Buxbaum, J. D., et al. (1990) Proc Natl Acad Sci USA 87:6003-6, which is incorporated herein by reference.

To measure Aβ peptides, or to measure APPₛ, which is the secreted carboxyl-terminal truncated form, cell supernatants are incubated with antibody 6E10, which recognizes the first 15 amino acids of the Aβ peptide that correspond to the COOH-terminal amino acids of APPₛ. See Buxbaum, J. D., et al. (1994) Proc Natl Acad Sci USA. 91:4489-93, which is incorporated herein by reference.

The incubations with antibody 6E10 or antibody 369 are performed at 4° C. for 75 minutes followed by a 45 minute incubation at 4° C. with either agarose-linked anti-mouse IgG for antibody 6E10 or protein A sepharose for antibody 369. The beads are then washed three times for 10 minutes and then run on either a 10-20% Tris-Ticine Gel for APPₛ and Aβ peptides or an 8% polyacrylamide get for cell-associated APP. The gels are dried and exposed to a Phosphor Imager® screen and exposed for a minimum of two days. The protein bands are visualized on a STORM 860 Phosphor Imager® (Molecular Dynamics) and quantitated using ImageQuant (Molecular Dynamics).

To measure extracellular APPₛ and fragments thereof, the cell culture supernatant are utilized. To measure the intracellular APPₗ and APPₘ and fragments thereof, the cell lysates are utilized.

The amount of extracellular Aβ peptides and the amount of extracellular APPₛ are normalized to the amount of total APPₗ found in the cell at the end of cell labeling as described above. This normalization provides an effective means of accounting for any differences between cultures and any differences due to altered APPᵢ synthesis or maturation in cells treated with the candidate compounds.

### Example 4

### Human Trials

A study was conducted to assess the effects of Lovastatin XL on blood lipid levels in patients with hyperlipidemia. Patients were treated with placebo, 10, 20, 40 or 60 mg per day of lovastatin administered as Lovastatin XL. Blood samples were obtained from selected patients prior to dosing and at 1 month after dosing. Because this clinical trial was carried out as part of an New Drug Application, at the time of filing U.S. Provisional Patent Application No. 60/223,987 (“the '987 application”), the inventors were not permitted to determine what dose (placebo, 10, 20, 40 or 60 mg) each of the selected patients where given per day. These blood samples were assayed for Aβ peptide concentrations (pg/ml). The results are listed in Table 9 below:

TABLE 9
Baseline and 1-Month Beta Amyloid Values

| | Baseline Aβ | Change in Aβ peptide |
|---|---|---|
| | peptide | conc. after 1 Month |
| Patient | conc. (pg/ml) | (pg/ml) |
| 1 | 145.2 | −57.8 |
| 2 | 211.1 | −30.5 |
| 3 | 151.1 | −16.9 |
| 4 | 175.5 | 60.6 |
| 5 | 388.1 | 44 |
| 6 | 499.7 | −172.7 |
| 7 | 164 | −64.1 |
| 8 | 220 | −67.5 |
| 9 | 215.5 | −80.3 |
| 10 | 370.1 | −18.5 |
| 11 | 403.9 | −76.2 |
| 12 | 48.9 | −32.9 |
| 13 | 15.6 | 38.4 |
| 14 | 64.6 | −13.4 |
| 15 | 34.3 | −18 |
| 16 | 12 | −1.1 |
| 17 | 45.4 | 10.9 |
| 18 | 12 | 4.6 |
| 19 | 37.3 | −5.6 |
| 20 | 30.6 | 0.2 |
| 21 | 35.2 | 10 |
| 22 | 138.5 | −112.5 |
| 23 | 60.6 | −38.5 |
| 24 | 50 | 22 |
| 25 | 73 | −22 |
| 26 | 133 | −17 |
| 27 | 23 | 70 |
| 28 | 82 | −21 |
| 29 | 4 | 4 |
| 30 | 56 | 5 |
| 31 | 59 | 17 |
| 32 | 2 | 4 |
| 33 | 3 | 1 |
| 34 | 181 | −42 |
| 35 | 0 | 5 |
| 36 | 175.4 | −37 |
| Mean | 120.016667 | −18.0222222 |
| SD | 126.784261 | 46.9449472 |

As can be seen from Table 9, the mean Aβ peptide concentration prior to treatment was 120 pg/ml, which decreased by about 18 pg/ml after one month of treatment with Lovastatin XL. The change from pre-treatment was statistically significant (p=0.0273) as shown in Table 10. In the instant case, one of ordinary skill in the art will understand that the above referred to p value represents the probability that the reported change in Aβ peptide concentration could occur by chance. One of ordinary skill in the art will also understand that a p value of less that 0.05 signifies that the reported change is statistically meaningful.

TABLE 10
Statistical Analysis of Change From Baseline

Results:
Two tailed T-test results:

| Variable | | MUO Estimate | Std. Err. | DF |
|---|---|---|---|---|
| varl | 0 | −18.022223 7.8241577 | 35 −2.3034072 | Tstat |
| Variable | Pval | | | |
| varl | 0.0273 | | | |

Following the filing of the '987 application, the inventors have performed a more detailed analysis of the data obtained from the human trials. Table 11 sets forth a dose-response analysis of the data and shows the mean percentage change in the Aβ peptide concentration in the blood of the patients treatment with placebo, 10, 20, 40 and 60 mg/day Lovastatin XL. Blood samples were taken from the patients after four weeks of treatment Study Visit No. 5), after eleven weeks of treatment (Study Visit No. 7) and twelve weeks of treatment with Lovastatin XL (Study Visit No. 8).

TABLE 11
Effect of Various Doses of
Lovastatin XL on Aβ Peptide Concentration

| | | | | Average of | |
|---|---|---|---|---|---|
| DOSE | | Study Visit | Study Visit | Study Visit | END- |
| (mg/day) | | No. 5 | No. 7 | Nos. 7 & 8 | POINT |
| 0 | N | 11 | 6 | 8 | 12 |
| | MEAN | −1.78 | −16.27 | −21.11 | −11.87 |
| | STD | 33.69 | 29.03 | 23.16 | 27.03 |
| 10 | N | 10 | 5 | 6 | 10 |
| | MEAN | 8.09 | 43.29 | 10.21 | −0.21 |
| | STD | 64.92 | 72.69 | 57.83 | 49.66 |
| 20 | N | 10 | 5 | 8 | 11 |
| | MEAN | 0.59 | −18.21 | −27.81 | −21.71 |
| | STD | 35.56 | 43.00 | 49.91 | 45.04 |
| 40 | N | 6 | 3 | 4 | 10 |
| | MEAN | −6.51 | −6.15 | −26.35 | −11.92 |
| | STD | 22.75 | 32.05 | 48.13 | 46.12 |
| 60 | N | 8 | 3 | 7 | 10 |
| | MEAN | −5.08 | −16.27 | −35.47 | −39.42 |
| | STD | 39.78 | 25.78 | 27.83 | 30.09 |

N—No. of patients
MEAN—mean % change in the Aβ peptide concentration in the blood of patients
STD—Standard deviation

The graph shown in FIG. 8 depicts the results set forth in Table 11 above. In particular, the graph shows the change in mean Aβ peptide concentration in the blood of patients after one month of treatment with Lovastatin XL as a function of the dose administered. FIG. 8 also includes a “trendline”, i.e., the best straight-line approximation, of the data presented in the graph. As can be seen from the “trendline”, its direction and slope clearly suggests that the dose of Lovastin XL administered does have an effect on the mean Aβ peptide concentration. The inventors also found that the dose-response analysis for the endpoint values was statistically significant (p=0.0442).

Further human trials have been conducted. Patients meeting the current criteria for treatment with lipid lowering agents were treated with single-blind placebo for 4 weeks. Those patients were then randomly assigned to receive daily doses of 10, 20, 40 or 60 mg/day of a controlled release lovastatin (Lovastatin XL) or matching placebo under double-blind conditions. Serum samples from those patients were obtained prior to and after 3 months of dosing. The serum samples were assayed for Aβ peptide using the assay set forth in Example 5 below. The assay results, expressed as percent change from pre-treatment of serum Aβ peptide concentration levels, are shown in FIG. 9.

As can be seen from FIG. 9, the placebo treated patients showed a mean increase of serum Aβ peptide concentration levels from baseline, however, this difference was not found to be statistically significant. The mean percent changes in the serum Aβ peptide concentration levels for the patients who received treatment with the controlled release formulation of lovastatin all decreased. These reported percentage changes for the groups of patients treated with 20, 40 and 60 mg/day were determined to be statistically significant p<0.001 (t-test). Further, the percentage changes for the groups of patients treated with 40 and 60 mg/day were determined to be statistically significantly different from those of the placebo group, p<0.05 (t-test).

### Example 5

### Aβ End-Specific Protocol

The serum samples referred to above in Example 4 were assayed for Aβ peptide using appropriate assays. The assay used for the human trials, the results of which are set forth in FIG. 9, was carried out as follows:

Ninety-six well plates (Falcon Probind) were coated with 150 μl of the 4G8 monoclonal antibody (Senetek Crude IgG Ascites Fluid) in carbonate-bicarbonate buffered solution (Sigma) and then incubated at 37° C. for 12-16 hours. The plates were then washed three times with 150 μl/well ECW buffer (PBS, 0.1% BSA, 0.05% Tween-20, 0.2% CHAPS, 5 mM ethylenediaminetetraacetic acid, 2 mM betaine, 0.05% NaN₃) before adding 150 μl/well ECW buffer containing 1% casein and incubated at 37° C. for an additional 4 hours. The 4G8 antibody recognizes Aβ and thus selects this peptide from the pool of others in the plasma.

The coated plates were washed twice with 150 μ/well ECW and then 50 μl/well ECW was added to ensure that the wells did not dry out during sample loading.

The standard curves of synthetic Aβ 1-40 peptide were prepared by diluting the 100 ng/μl into working solutions using ECW. For the assays the following concentrations were used: 0, 10, 50, 100, 250 and 500 pg/ml synthetic peptide. The standards were loaded in duplicate onto the wells.

Each plasma sample was thawed and then sonicated for 20 seconds prior to loading onto wells in quadruplicate. Each plate contained two internal reference samples and all patient visits were loaded on the same plate.

The loaded plates were incubated for 5 minutes at room temperature, and then for 2 days at 4° C. (“capture phase”). The plates were then washed twice with 150 μl/well ECW, 150 μl of biotinylated 6E10 monoclonal antibody (Senetek mAbs Biotin 6E10) diluted 1:1000 in ECW was added to each well and the plates incubated at room temperature for 12-15 hours. The 6E10 antibody recognizes Aβ which is “captured” by the 4G8 and is biotinylated so that it can be detected by the tertiary antibody.

The plates were washed three times with 150 μl/well ECW before 150 μl of streptavidin alkaline phophatase (Amersham) was added per well and incubated at room temperature for 5 hours. The plates were then washed three times with 150 μl/well ECW before 100 μl/well ddH₂O was added. The water was then aspirated and 100 μl of the Attophos reagent (JBL Scientific Inc) added per well before being allowed to develop at room temperature in the dark. When the highest point in the standard curve began to turn yellow, the plates were read on a microplate reader (PerSeptive Biosystems CytoFluor Series 4000) at an excitation of 450 nm and an emission of 530 nm.

### Example 6

### Results for Percent Change from Baseline to Averaged Last Observation

Table 12 displays the results for the % Change from Baseline to the Averaged Last Observation.

For this dataset, the percent change from baseline was calculated for each plate using the value from each plate for the last for the last available visit. For each subject, the value used for the % change from baseline to last observation was the average of all the plates for that subject.

TABLE 12
Percent Change from Baseline to Averaged Last Observation

| | | | Mean (SEM) | p-value | p-value | Dunnett's p- |
|---|---|---|---|---|---|---|
| | | Median % | % Change | (Change | (pairwise | value |
| Dose | | Change from | from | from | comparison | (comparison |
| Group | n | Baseline | Baseline | Baseline) | to placebo) | to placebo) |
| Placebo | 15 | −1.1 | 37.4 (30.2) | 0.236 | — | — |
| 10 mg | 20 | −5.9 | 14.1 (12.6) | 0.275 | 0.293 | 0.647 |
| 20 mg | 19 | −14.0 | .06 (10.2) | 0.955 | 0.102 | 0.276 |
| 40 mg | 20 | −38.6 | −23.6 (10.7) | 0.039 | 0.0068 | 0.023 |
| 60 mg | 20 | −31.7 | −21.3 (10.3) | 0.052 | 0.0092 | 0.031 |

The results in Table 12 indicate that treating patients with lovastatin XL produced dose-Dependent and statistically significant decreases in serum β-amyloid concentrations. A statistically significant change from baseline was observed in the 40 mg dose group, and the changes from baseline for both the 40 and 60 mg dose groups were significantly different from placebo by both pairwise comparisons and Dunnett's test (Table 12). The overall treatment effect displayed in Table 12 was statistically significant when evaluated by ANOVA (p<0.0348), ANCOVA, Kruskal-Wallis (p<0.0098), and ANOVA on the ranked data (p<0.0076).

Certain of the above disclosure is presented with respect to HMG-CoA reductase inhibitors. As other agents which lower A β levels are contemplated in the present invention, (e.g., NSAIDS), the disclosure with respect to HMG-CoA reductase inhibitors is applicable to these agents as well.

The foregoing description has been presented for purposes of illustration and description. It is not intended to be exhaustive or to limit the invention. The accompanying drawings are included to provide a further understanding of the invention and are incorporated in and constitute a part of this specification, illustrate several embodiments of the invention and together with the description serve to explain the principles of the invention. Obvious modifications or variations are possible in light of the above teachings. All such obvious modification and variations are intended to be within the scope of the present invention.

What is claimed is:

1. A method for managing a patient with Alzheimer's disease or at risk of developing Alzheimer's disease comprising:
providing to said patient a therapeutic agent which lowers Aβ levels, and detecting a level of Aβ in a body fluid of said patient to determine the efficacy of said therapeutic agent.

2. The method of claim 1 wherein said therapeutic agent is an HMG CoA reductase inhibitor, an NSAID, a secretase modifier or a combination thereof.

3. The method of claim 1, further comprising repeatedly detecting the level of Aβ in a body fluid.

4. The method of claim 1, further comprising repeatedly providing said therapeutic agent according to a dosing interval.

5. The method of claim 4, further comprising repeatedly detecting the level of Aβ in a body fluid.

6. The method of claim 5, further comprising comparing a detected level of Aβ in said body fluid with at least one previously detected level of Aβ.

7. The method of claim 6, further comprising adjusting the repeated dosing of said therapeutic agent based on said comparison.

8. The method of claim 1, wherein said body fluid is blood plasma or serum.

9. The method of claim 1, wherein said therapeutic agent is an HMG-CoA reductase inhibitor.

10. The method of claim 9, wherein said HMG-CoA reductase inhibitor is selected from the group consisting of mevastatin, pravastatin, simvastatin, atorvastatin, lovastatin, rivastatin, fluvastatin, pharmaceutically acceptable salts thereof, isomers thereof, and active metabolite thereof.

11. The method of claim 9, wherein said HMG-CoA reductase inhibitor is lovastatin or a pharmaceutically acceptable salt thereof.

12. The method of claim 9, wherein said HMG-CoA reductase inhibitor is in a controlled release oral dosage form.

13. The method of claim 1, wherein said levels of Aβ are detected in said body fluid using an assay.

14. The method of claim 1, wherein said assay selected from the group consisting of radioimmunoassays, ELISA (enzyme linked immunosorbent assay), “sandwich” immunoassays, precipitin reactions, gel diffusion precipitin reactions, immunodiffusion assays, agglutination assays, complement-fixation assays, immunoradiometric assays, fluorescent immunoassays, western blots, protein A immunoassays, and immunoelectro-phoresis assays, and combinations thereof.

15. The method of claim 13, wherein said assay is an ELISA.

16. The method of claim 1, further comprising detecting a baseline level of Aβ prior to providing said therapeutic agent.

17. A kit for an assay of Aβ serum concentration comprising a capture-antibody and a detection-antibody, wherein said antibodies do not bind to overlapping epitopes of the Aβ and wherein said antibodies allow specific detection of the Aβ.

18. The kit of claim 17, wherein said capture-antibody is bound to a solid support and said detection-antibody is coupled with a label.

19. The kit of claim 17, wherein the capture-antibody is an anti-Aβ antibody that binds to an epitope between residues 17 and 20 of said Aβ and the detection-antibody is an anti-Aβ antibody that binds to an epitope at about residue 11.

20. The kit of claim 17, wherein said antibodies are monoclonal antibodies.

21. The kit of claim 17, further comprising a binding buffer, a wash buffer, a detection buffer, a control standard and label detection means.

22. A method for treating, preventing or inhibiting an APP processing disorder in a mammal comprising administering to said mammal a controlled release composition comprising an effective amount of at least one HMG-CoA reductase inhibitor to lower Aβ levels.

23. The method of claim 22, wherein said method comprises lowering the amount of Aβ peptide in the brain, cerebral spinal fluid, or plasma.

24. The method of claim 22, wherein lowering the amount of Aβ peptides in the brain comprises affecting APPₘ processing.

25. The method of claim 22, wherein the APP processing disorder is Alzheimer's Disease or Down's Syndrome.

26. The method of claim 22, wherein the HMG-CoA reductase inhibitor is selected from the group consisting of mevastatin, pravastatin, simvastatin, atorvastatin, lovastatin, rivastatin, fluvastatin, and pharmaceutically acceptable salts, isomers and active metabolite forms thereof.

27. The method of claim 22, wherein the HMG-CoA reductase inhibitor is lovastatin or lovastatin acid.

28. The method of claim 27, wherein up to 240 mg of the HMG-CoA reductase inhibitor is administered per day.

29. The method of claim 27, wherein about 10 to about 120 mg of the HMG-CoA reductase inhibitor is administered per day.

30. The method of claim 27, wherein about 10 mg to about 60 mg of the HMG-CoA reductase inhibitor is administered per day.

31. The method of claim 22, wherein about 0.2 mg to about 10 mg of the HMG-CoA reductase inhibitor per Kg of the mammal's body weight is administered per day.

32. The method of claim 22, wherein the composition comprises an amount of the HMG-CoA reductase inhibitor such that the average blood plasma concentration of the HMG-CoA reductase inhibitor or an active metabolite thereof at steady-state is below about 50 nanomolar.

33. The method of claim 22, wherein said lowering Aβ results from increasing the clearance of Aβ peptides.

34. A method for treating, preventing or inhibiting an APP processing disorder in a mammal comprising administering to said mammal a controlled release composition comprising an effective amount of at least one HMG-CoA reductase inhibitor to prevent or reduce Aβ peptide aggregation or plaque formation in the brain of the mammal.

35. The method of claim 34, wherein the HMG-CoA reductase inhibitor decreases the formation of Aβ peptides, increases the clearance of Aβ peptides, regulates the processing of APP, or reduces plaque maturation in the mammal.

36. The method of claim 34, wherein the APP processing disorder is Alzheimer's disease and the method slows the progression of Alzheimer's disease.

37. The method of claim 34, wherein detected Aβ levels are decreased by about 5% or more

38. A method for treating, preventing or inhibiting an APP processing disorder in a mammal comprising administering a composition which lowers the amount of cellular cholesterol levels in the mammal.

39. A method for managing a patient with Alzheimer's disease or at risk of developing Alzheimer's disease comprising:
detecting a level of Aβ in a body fluid of a patient receiving HMG-CoA reductase inhibitor therapy.

40. The method of claim 39, further comprising adjusting said HMG-CoA reductase inhibitor therapy based on said Aβ level.
